(19) United States
(12) Patent Application Publication
(10) Pub. No.: US 2024/0321430 A1
(43) Pub. Date: Sep. 26, 2024

(54) METHOD AND SYSTEM FOR NEUROMODULATION

(71) Applicant: xSensa Labs, Toronto (CA)

(72) Inventor: Natasha Kovacevic, Toronto (CA)

(21) Appl. No.: 18/638,043

(22) Filed: Apr. 17, 2024

Related U.S. Application Data
(63) Continuation of application No. 17/487,309, filed on Sep. 28, 2021, now abandoned.
(60) Provisional application No. 63/084,121, filed on Sep. 28, 2020.

(51) Int. Cl. G16H 20/70; A61B 5/00; A61B 5/16; A61B 5/246; A61B 5/374

(52) U.S. Cl. CPC G16H 20/70; A61B 5/0075; A61B 5/165; A61B 5/246; A61B 5/374; A61B 5/7257

(57) ABSTRACT

A method and system for neuromodulation therapy is described. One or more psychophysiological parameters based on an electrical signal of a user's brain activity are determined and used to generate a neuromodulation marker using a neuromodulation recipe. The neuromodulation marker may be determined using statistical estimates of the psychophysiological parameters in relation to a reference distribution. Feedback is then provided to the user based on the neuromodulation marker. Weighting parameters may be used to vary the neuromodulation recipe and personalize the neuromodulation therapy in response to performance or user input.

### CROSS-REFERENCE TO RELATED APPLICATIONS

This application is a continuation of U.S. patent application Ser. No. 17/487,309, filed Sep. 28, 2021, which claims priority from U.S. Provisional Patent Application No. 63/084, 121 filed Sep. 28, 2020, the entire contents of which are hereby incorporated by reference.

### FIELD

The described embodiments relate to a method and system for neuromodulation and more specifically to a method and system for self-directed active neuromodulation for improving cognitive, emotional, or behavioral performance.

### BACKGROUND

Wearable wireless brain-sensing devices on the consumer market have created an opportunity to positively impact mental health and wellbeing and shift from a reactive, remedial mindset to one that is pro-active, preventative and enhancement oriented. Availability of low-cost sensors, wide penetration of mobile devices and ubiquitous access to the internet are making it possible to empower the individual, increasing self-reliance and reducing the dependence on the health care system.

A surge of new brain-computer interface technologies (BCI) has brought forth a host of innovative brain-powered systems in entertainment (such as brain-controlled video games), niche applications (such as communication with patients in a locked-in state, motor-rehabilitation, alleviation of ADHD symptoms), or more general tools such as technology-aided meditation. Health-related technologies that employ non-invasive brain sensors and promote natural abilities to activate and strengthen specific brain functional patterns using principles of neurofeedback (known as self-directed active neuromodulation or SDAN) comprise the largest BCI sector.

BCI technologies face technical challenges with the use of non-invasive brain sensors. This can include the significant amount of data generated during brain activity monitoring. BCI technologies may further operate on mobile devices, or as an embedded system in a device, which may have limited processing resources and thus exacerbate the processing challenges associated with the brain sensor data. BCI technologies such as those for use in non-clinical settings may further operate on brain sensing devices with limited number of sensors and no shielding from electrical interference from surrounding environments, and thus exacerbate the processing challenges associated with the brain sensor data.

Commercially available non-invasive brain sensors suffer from sub-optimal signal quality and nuisance variability issues, and BCI technologies face technical problems in terms of sensor data quality and sensor reliability. Applications relying on sub-optimal sensor data may produce unreliable output and may be ineffective for their stated goals.

While a number of solutions have been proposed for cognitive health and performance, mood, emotional regulation, athletic performance, creativity, and productivity, the field is lacking solid evidence of promised benefits, even in narrowly focused applications. Several meta-analyses and comparative reviews have identified methodological challenges, lack of reproducibility and even contradictory findings from different groups (Cortese et al., 2016; Sitaram et al. 2017; Omejc et al., 2019; Ali et al., 2020). There are very few solutions that report ‘transfer effects’ or ‘generalizable effects’, that is, demonstrative gains in mental capacities measured by tests that the user has not trained for. Of those, only a handful have employed rigorous randomized control double-blind design with control for placebo (Wang and Hsieh, 2013; Xiong et al., 2014; Schabus et al., 2017; Brandmeyer and Delorme, 2020), all of which were performed in research labs, under supervision, using expensive equipment, and a relatively small number of subjects (less than 35). Beyond-placebo transfer effects with consumer-grade brain sensors in real-life scenarios have not been reported thus far.

Current state-of-the-art wearable EEG devices have shown stable test-retest reliability under supervision in lab environments (Rogers et al. 2018). In real-life applications, however, EEG data quality is considerably more susceptible to artifacts. There remains a need for methods and systems for self-directed active neuromodulation that are adaptable and can produce transfer effects across a diverse population of subjects.

### SUMMARY

This summary is intended to introduce the reader to the more detailed description that follows and not to limit or define any claimed or as yet unclaimed invention. One or more inventions may reside in any combination or sub-combination of the elements or process steps disclosed in any part of this document including its claims and figures.

In one aspect of this disclosure, there is provided a method and system for neuromodulation, otherwise known as neurofeedback training or neuromodulation therapy. In one embodiment, the method and system may be used for self-directed active neuromodulation (SDAN).

In one embodiment, the methods and systems described involve acquiring subject brain data, processing subject brain data to determine a neuromodulation marker, and communicating feedback to the subject based on the neuromodulation marker. The subject may then modulate their brain activity, and accordingly the neuromodulation marker, in time through a mental process. The feedback may be determined based on the neuromodulation marker and optionally include a relative difference or similarity of the neuromodulation marker to a target or difficulty threshold.

Using particular neuromodulation markers based on one or more psychophysiological parameters may reduce the amount of data required for processing, However, it is challenging to determine the particular parameters for inclusion since they should desirably both accurate and limited in number to reduce processing overhead. In one aspect, the embodiments described herein provide for the efficient and accurate analysis of psychophysiological parameters for measurement based on several identified markers thereby advantageously reducing the processing of the system and method to a subset of the sensor data collected. In one embodiment, this may improve the responsiveness and the communication of feedback to a subject during neuromodulation and in particular for SDAN.

One aspect of the present disclosure involves the use of recipes or functions for determining the neuromodulation marker. The neuromodulation marker may be determined based on the amplitude of one or more psychophysiological parameters that are representative of the subject's brain activity. For example, in one embodiment the psychophysiological parameter is an amplitude value representative of a characteristic of the electrical signal of the subject's brain activity such as a spectral power, entropy, coherence, phase synchronization, asymmetry, correlation or other relationship between different electrode sites, spectral coupling, or cross-frequency correlation. In one embodiment, the psychophysiological parameter is amplitude of the relative or absolute spectral power associated with a frequency band.

In one embodiment, each psychophysiological parameter is associated with a weighting parameter such that the relative contribution of each psychophysiological parameter to the neuromodulation marker can be adjusted.

Another aspect of the present disclosure involves the use of statistical estimates of the psychophysiological parameters for determining the neuromodulation marker, optionally statistical estimates of the amplitude of the psychological parameters. For example, in one embodiment a percentile value for the amplitude of a psychophysiological parameter relative to a reference distribution is determined and then used for calculating the neuromodulation marker. The reference distribution may be a baseline distribution of the psychophysiological parameter determined at the start of a therapy session for the subject or during a therapy session for the subject.

In another embodiment, a statistical estimate of the neuromodulation marker is determined relative to a reference distribution and the statistical estimate of the neuromodulation marker is then used to provide feedback to the subject. For example, in one embodiment a percentile value for a neuromodulation marker is determined relative to a reference distribution, and this percentile value is used to provide feedback to the subject. The reference distribution may be a baseline distribution for the neuromodulation marker determined at the start of a therapy session for the subject or during a therapy session for the subject.

As shown in Example 1, neuromodulation therapy using statistical estimates (percentile values) of a single EEG parameter relative to a user baseline resulted in significant transfer effects and improved cognitive functioning in older adults. Furthermore, as shown in Example 2, effective neuromodulation therapy was achieved using statistical estimates of multiple EEG parameters and neuromodulation recipes that allow for the weighted combination of multiple measures into a single neuromodulation marker. Notably, feedback to the user was also based on a statistical estimate (percentile) of this single neuromodulation marker relative to a baseline distribution of the neuromodulation marker from the subject at the start of each session.

Without being limited by theory, the use of statistical estimates rather than raw values is thought to improve robustness of the method in regards to the issues of sub-optimal signal quality and nuisance variability; issues that are significant and unavoidable in mass-market applications using commercial EEG sensors. This robustness advantageously provides a technical solution to the challenges faced by BCI technologies due to sub-optimal signal quality and nuisance variability issues. As a result, the feedback to the user remains consistent from session to session, regardless of variations in the underlying raw EEG characteristics, and thus enables unsupervised self-administration of the method. In addition, the use of neuromodulation recipes and weighting parameters as described herein more universally captures therapeutic goals regardless of within-subject and across-subject variations of absolute measures of brain activity.

Accordingly, in one embodiment there is provided a method for providing neuromodulation therapy to a subject. In one embodiment, the method comprises:
(a) receiving, at a processor from a sensor in communication with the processor, a first electrical signal of the subject's brain activity during a first time interval;
(b) selecting, at the processor, one or more psychophysiological parameters of the first electrical signal;
(c) determining, at the processor, the one or more psychophysiological parameters based on the first electrical signal;
(d) determining, at the processor, an amplitude value of each of the one or more psychophysiological parameters;
(e) determining, at the processor, a first neuromodulation marker based on the amplitude values of the one or more psychophysiological parameters during the first time interval; and
(f) outputting, at an output device in communication with the processor, an output signal, the output signal comprising a first output property determined based on the first neuromodulation marker.

In one embodiment, the method further comprises:
(a) receiving, at the processor from the sensor, a second electrical signal of the subject's brain activity during a second time interval;
(b) selecting, at the processor, one or more psychophysiological parameters of the second electrical signal;
(c) determining, at the processor, the one or more psychophysiological parameters based on the second electrical signal;
(d) determining, at the processor, an amplitude value of each of the one or more psychophysiological parameters;
(e) determining, at the processor, a second neuromodulation marker based on one or more amplitude values of the one or more psychophysiological parameters during the second time interval; and
(f) providing feedback to the subject, at the output device in communication with the processor, by changing the output signal to a second output property determined based on the second neuromodulation marker.

In one embodiment, the one or more psychophysiological parameters of the first electrical signal may be the same type as the one or more psychophysiological parameters of the second electrical signal. In one embodiment, the output signal provided as feedback may be determined based on the difference between the first neuromodulation marker and the second neuromodulation marker. In one embodiment, the output signal provided to the user as feedback may be determined based on the difference between a difficulty threshold and at least one of the first neuromodulation marker and the second neuromodulation marker.

In one embodiment, the first neuromodulation marker or the second neuromodulation marker may be based on two or more amplitude values of two or more psychophysiological parameters.

In one embodiment, the method may further comprise determining at least one of the first neuromodulation marker and the second neuromodulation marker based on a statistical estimate of the one or more amplitude values.

In one embodiment, the method may further comprise determining the statistical estimate of the one or more amplitude values relative to a reference distribution of amplitude values of the psychophysiological parameter, optionally wherein the statistical estimate is a percentile value relative to the reference distribution of amplitude values.

In one embodiment, the reference distribution of amplitude values of the psychophysiological parameter may be based on aggregated subject data from one or more different subjects stored in a database.

In one embodiment, the reference distribution of the amplitude values of the psychophysiological parameter may be based on historical data of the subject. In one embodiment, the reference distribution of amplitude values of the psychophysiological parameter is a baseline distribution obtained from the user, optionally a baseline distribution obtained at the start of a neuromodulation therapy session or during a neuromodulation therapy session. In one embodiment, the method comprises receiving, at the processor from a sensor in communication with the processor, a baseline electrical signal of the subject's brain activity and determining, at the processor a reference distribution for a psychophysiological parameter based on the baseline electrical signal.

In one embodiment, each amplitude value of the psychophysiological parameters may be associated with a weighting parameter, and the method may further comprise determining at least one of the first neuromodulation marker and the second neuromodulation marker based on a weighted statistical estimate of the one or more amplitude values.

In one embodiment, determining at least one of the first neuromodulation marker and the second neuromodulation marker may be based on an additive combination of weighted statistical estimates of the two or more amplitude values.

In one embodiment, the method may further comprise adjusting the one or more weighting parameters during a neuromodulation therapy session or between neuromodulation therapy sessions.

In one embodiment, the method may further comprise determining, at the processor, one or more adjusted weighting parameters based on user input and/or subject data.

In one embodiment, the method may further comprise selecting, at the processor, the one or more psychophysiological parameters based on user input and/or subject data.

In one embodiment, the method may further comprise:
(a) providing, at a memory in communication with the processor, a subject database; and
(b) storing subject data in the subject database, wherein the subject data comprises electrical signal data, psychophysiological parameter data, amplitude value data, neuromodulator marker data, subject testing data or subject annotation data.

In one embodiment, determining the first output property or the second output property may be based on a statistical estimate of the first neuromodulation marker or the second neuromodulation marker relative to a reference distribution of the first neuromodulation marker or second neuromodulation marker respectively.

In one embodiment, the reference distribution of at least one of the first neuromodulation marker and second neuromodulation marker may be based on aggregated subject data from one or more different subjects stored in a database.

In one embodiment, the reference distribution of at least one of the first neuromodulation marker and second neuromodulation marker may be based on historical data of the subject. In one embodiment, the method comprises receiving, at the processor from a sensor in communication with the processor, a baseline electrical signal of the subject's brain activity and determining, at the processor a reference distribution for the neuromodulation marker based on the baseline electrical signal, optionally a reference distribution for the first and/or second neuromodulation marker. In one embodiment, a reference distribution of at least one of the first neuromodulation marker and second neuromodulation marker is determined at the start of a neuromodulation therapy session for a subject.

In one embodiment, the sensor may comprise an electroencephalogram (EEG) sensor, a functional magnetic resonance imaging (fMRI) sensor, a magnetoencephalography (MEG), or a functional Near Infrared Spectroscopy (fNIRS) sensor. In one embodiment, the method further comprises positioning the sensor on the head of the subject.

In one embodiment, the sensor may comprise the EEG sensor and first electrical signal and second electrical signal may comprise voltage data, and optionally multichannel voltage data.

In one embodiment, the psychophysiological parameters may be determined based on a Fourier transform of the first electrical signal or the second electrical signal. In one embodiment, the amplitude values of each of the psychophysiological parameters may be spectral power values. In one embodiment, the amplitude value of the psychophysiological parameters is a measure of entropy, coherence, phase synchronization, asymmetry, correlation between different electrode sites, spectral coupling, or cross-frequency correlation for the electrical signals of the subject's brain activity.

In one embodiment, the spectral power values of the one or more psychophysiological parameters may be determined from one or more frequency bands of the first electrical signal or second electrical signal. For example, in one embodiment, the one or more frequency bands may comprise at least one of an alpha band, a beta band, a gamma band and a theta band. In one embodiment, the first neuromodulation marker or the second neuromodulation marker may be determined based on at least one frequency band selected from the group consisting of the alpha band, the beta band, the gamma band, and the theta band.

In one embodiment, the output signal to the subject may be determined based on a temporal smoothing over a predetermined time interval of a plurality of neuromodulation markers comprising at least the first neuromodulation marker and the second neuromodulation marker.

In one embodiment, the temporal smoothing may be based on a moving weighted average with a window length of between 0.2 and 2 seconds, optionally between 0.5 and 1.5 seconds or about 1 second. In one embodiment, the temporal smoothing is based on an overlap between consecutive windows of between 25 ms and 1 second, optionally between about 100 ms and 500 ms or about 250 ms between consecutive windows.

In one embodiment, the output signal to the subject may be determined by comparing the first and/or second neuromodulation marker to a difficulty threshold.

In one embodiment, the method may further comprise receiving, at a user input device in communication with the processor, a user input, optionally from the subject, and adjusting, at the processor, the difficulty threshold based on the user input.

In one embodiment, the method may further comprise adjusting, at the processor, the difficulty threshold based on the first and/or second neuromodulation marker, optionally based on the subject's neuromodulation performance over a pre-determined time interval or recent historical values.

In one embodiment, the method may further comprise:
(a) transmitting, from a network device in communication with the processor to a remote server, subject data, wherein subject data comprises at least one selected from the group of electrical signal data, psychophysiological parameter data, amplitude value data, neuromodulator marker data, subject testing data or subject annotation data;
(b) receiving, at the network device from the remote server, at least one selected from the group of a revised difficulty threshold and one or more adjusted weighting parameters; and
(c) adjusting, at the processor, the difficulty threshold or weighting parameters based on the revised difficulty threshold or adjusted weighting parameters, respectively.

In one embodiment, the method may further comprise:
(a) transmitting, from a network device in communication with the processor to a remote server, subject data, wherein subject data comprises at least one selected from the group of electrical signal data, psychophysiological parameter data, amplitude value data, neuromodulator marker data, subject testing data or subject annotation data;
(b) receiving, at the network device from the remote server, at an adjusted recipe for determining the neuromodulation marker; and
(c) adjusting, at the processor, the recipe for determining the neuromodulation marker.

In one embodiment, the output device may comprise at least one selected from the group of a display device, an audio output device, and a tactile device and the output signal may comprise at least one selected from the group of a visual output displayed on the display device, an audio output to the audio output device, and a tactile output to the tactile device.

In one embodiment, the output signal may activate two output devices, optionally cross-modal output devices.

In one embodiment, the method may further comprise providing a video game application to the subject using the processor and the output device, the video game comprising the output signal.

In one embodiment, the method may further comprise providing an interactive soundscape application to the subject using the processor and the output device, the interactive soundscape application comprising the output signal.

In one embodiment, the method may further comprise providing a meditation application to the subject using the processor and the output device.

In one embodiment, the method may further comprise:
(a) providing a cognitive testing application to the subject using the processor and the output device; and
(b) storing subject cognitive testing data in a subject database.

In one embodiment, the method may further comprise:
(a) providing a self-evaluation application to the subject using the processor and the output device; and
(b) storing subject self-evaluation data in a subject database.

In one embodiment, the method may be for the treatment of a condition or disease in a subject in need thereof. For example, in one embodiment the method is for the treatment of one or more of cognitive impairment, memory loss, depression, anxiety, post-traumatic disorder, obsessive-compulsive disorder, addiction, sleep disorder and attention deficit disorder in the subject. In one embodiment, the method may be for improving at least one or more of cognitive performance, attention, emotional regulation, mood enhancement, resilience to stress, impulsivity regulation, creativity, motivation, memory and well-being in the subject.

In accordance with one aspect, there is also provided a system for providing neuromodulation therapy according to a method as described herein.

In one embodiment, the system comprises:
(a) at least one sensor;
(b) a memory;
(c) a processor in communication with the at least one sensor and the memory, the processor configured to:
   (i) receive from the at least one sensor a first electrical signal of the subject's brain activity during a first time interval;
   (ii) select one or more psychophysiological parameters of the first electrical signal;
   (iii) determine the one or more psychophysiological parameters based on the first electrical signal;
   (iv) determine an amplitude value of each of the one or more psychophysiological parameters;
   (v) determine a first neuromodulation marker based on one or more amplitude values of the one or more psychophysiological parameters during the first time interval; and
   (vi) determine a first output property based on the first neuromodulation marker.

In one embodiment, the system further comprises: d) an output device, the output device for outputting an output signal, the output signal comprising the first output property. In one embodiment, the system further comprises a sensor for measuring the first or second electrical signal of the subject's brain activity. In one embodiment, the sensor is an EEG.

In one embodiment, the processor may be further configured to:
(i) receive from the at least one sensor a second electrical signal of the subject's brain activity during a second time interval;
(ii) select one or more psychophysiological parameters of the second electrical signal;
(iii) determine the one or more psychophysiological parameters based on the second electrical signal;
(iv) determine an amplitude value of each of the one or more psychophysiological parameters of the second electrical signal;
(v) determine a second neuromodulation marker based on one or more amplitude values of the one or more psychophysiological parameters during the second time interval; and
(vi) determine a second output property based on the second neuromodulation marker, and
wherein the output device provides feedback to the subject by changing the output signal to the second output property.

It will be appreciated by a person skilled in the art that an apparatus or method disclosed herein may embody any one or more of the features contained herein and that the features may be used in any particular combination or sub-combination.

These and other aspects and features of various embodiments will be described in greater detail below.

### BRIEF DESCRIPTION OF THE DRAWINGS

For a better understanding of the described embodiments and to show more clearly how they may be carried into effect, reference will now be made, by way of example, to the accompanying drawings in which:

FIG. 1, in a flow chart, illustrates one embodiment of a method of providing neuromodulation therapy;

FIG. 2 illustrates a feedback loop for neuromodulation therapy;

FIG. 3 shows example system for providing neuromodulation therapy;

FIG. 4 shows two examples of r-values (neuromodulation markers) obtained with different weighting parameters;

FIG. 5 shows an example processing pipeline within the frontend computing device and comprising two EEG metrics X and Y;

FIG. 6 is a graphical representation of a training session having a modular structure;

FIG. 7 is an illustration of an example of annotated brain-behavior for one subject performed at two different time points for the same activity, a Spatial Memory Task;

FIG. 8 is a schematic example of an annotated database;

FIG. 9 is a graphical representation of a single neuromodulation therapy session;

FIG. 10 is an example of a report of trends in wellbeing metrics following neuromodulation therapy;

FIG. 11 is an example report provided to a subject of transfer effects following neuromodulation therapy;

FIGS. 12A-C show screen shots of the user interface spatial memory task incorporated in the Memoride application used for neuromodulation therapy;

FIGS. 13A-C show results from the Attention Network Task (ANT) for the TNF and RNF (control) groups Pre and Post neuromodulation therapy;

FIGS. 14A-B show results from the verbal fluency test (FAS) for the TNF and RNF (control) groups Pre and Post neuromodulation therapy;

FIG. 15 is an example of a user interface for providing sensory feedback.

FIG. 16 shows screen shots of the user interface used in the application to determine self-reported wellness metrics;

FIG. 17 shows longitudinal wellness data of a subject over 18 neuromodulation therapy sessions as described in Example 2;

FIG. 18 shows a heat map for correlations between Z-scored slopes after regressing out stress for wellness metrics and neuromodulation therapy performance metrics.

The drawings included herewith are for illustrating various examples of articles, methods, and apparatuses of the teaching of the present specification and are not intended to limit the scope of what is taught in any way.

### DESCRIPTION OF EXEMPLARY EMBODIMENTS

Various apparatuses will be described below to provide an example of an embodiment of each claimed invention. No embodiment described below limits any claimed invention and any claimed invention may cover apparatuses that differ from those described below. The claimed inventions are not limited to apparatuses having all of the features of any one apparatus described below or to features common to multiple or all of the apparatuses described below. It is possible that an apparatus described below is not an embodiment of any claimed invention. Any invention disclosed in an apparatus described below that is not claimed in this document may be the subject matter of another protective instrument, for example, a continuing patent application, and the applicants, inventors or owners do not intend to abandon, disclaim or dedicate to the public any such invention by its disclosure in this document.

The terms “an embodiment”, “embodiment”, “embodiments”, “the embodiment”, “the embodiments”, “one or more embodiments”, “some embodiments”, and “one embodiment” mean “one or more (but not all) embodiments of the present invention(s),” unless expressly specified otherwise.

The terms “including”, “comprising”, and variations thereof mean “including but not limited to”, unless expressly specified otherwise. A listing of items does not imply that any or all of the items are mutually exclusive, unless expressly specified otherwise. The terms “a”, “an”, and “the” mean “one or more”, unless expressly specified otherwise.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers (referred to below as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and combination thereof.

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloadings, magnetic and electronic storage media, digital and analog signals, and the like. The computer useable instructions may also be in various forms, including compiled and non-compiled code.

### Neuromodulation Therapy

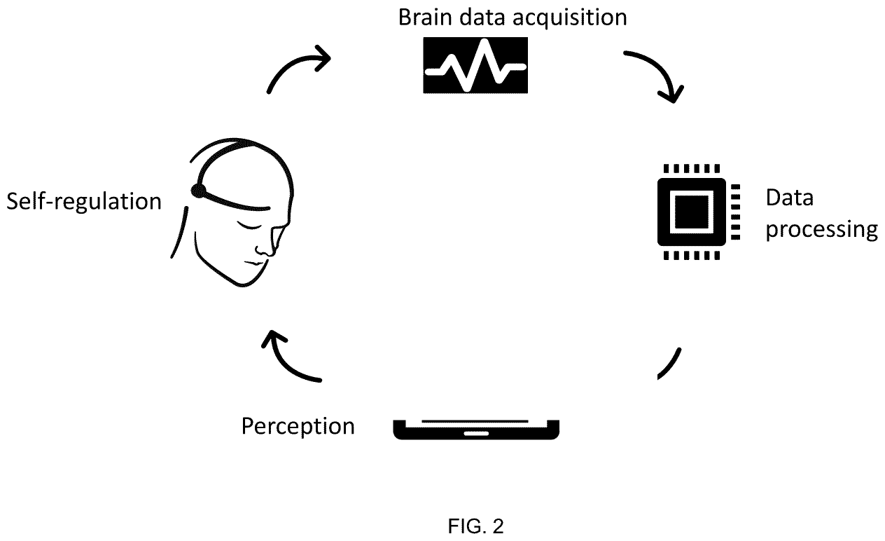

Neuromodulation therapy, otherwise known as neurofeedback training, is a closed-loop training system for self-regulation of brain activity via operant conditioning (i.e., a type of associative learning process through which the strength of a behaviour is modified by reinforcement (i.e., reward) or punishment). As shown in FIG. 2, neuromodulation therapy may operate as a feedback loop, that can be broken into 4 phases: (1) brain data acquisition; (2) data processing and extraction of a parameter of interest (referred to herein as a neuromodulation marker); (3) perception, where the subject receives sensory feedback based on the neuromodulation marker (usually visual and/or auditory feedback); and (4) self-regulation, a mental process through which the subject modulates the feedback in a therapeutic direction.

In the brain data acquisition phase, a system may continuously monitor a subject's brain activity, using any brain imaging modality known in the art (for example, an electroencephalogram (EEG) that collects data from at least one electrode location, functional magnetic resonance imaging (fMRI), magnetoencephalography (MEG), or functional near-infrared spectroscopy (fNIRS)). The raw data collected by the brain imaging modality sensor can be electronically or wirelessly transmitted to a processing unit, which may be, for example, a computer or a mobile device.

In the data processing phase, a subset of the raw data, i.e., a portion of the continuous stream of data defined by a period of time, is processed to obtain a neuromodulation marker (i.e. an indicator of brain activity) that can be communicated to the subject providing feedback to the user on their brain activity. Optionally, the subset of data may first be pre-processed to e.g. filter or correct for artifacts or rejected if excess artifacts or noise are present.

Acceptable data may then be processed to identify or select one or more psychophysiological parameters. For example, raw data in the form of EEG data may be processed to identify known frequency bands of brainwaves contained in the raw data (e.g., at least one of delta, theta, alpha, beta, and gamma bands).

In addition to identifying the one or more psychophysiological parameters within the subset of data during the processing phase, qualities of the psychophysiological parameters may also be determined. For example, the amplitudes of the frequency bands may be determined. In one embodiment, a statistical estimate of the amplitude of a psychophysiological parameter is determined, such as by determining a percentile value relative to a reference distribution of amplitude values.

In one aspect of the disclosure, a neuromodulation marker is determined based on one or more psychophysiological parameters. The neuromodulation marker may be based on two or more amplitude values of two or more psychophysiological parameters. It is also to be understood that the neuromodulation marker may be based on a single amplitude value of a single psychophysiological parameter.

The processing phase may also include comparing the neuromodulation marker for the subset of data to other, previously determined, neuromodulation markers such as a threshold (reference) neuromodulation marker value or distribution of values. The previously determined neuromodulation markers may be stored in the system and may represent optimal or target neuromodulation marker values. Alternatively, the previously determined neuromodulation markers may be neuromodulation markers determined from a prior subset of data from the same subject during the same training session or from multiple sessions.

In the perception phase, the data generated during the processing phase, such as, for example, the neuromodulation marker, may be presented to the subject via a neurofeedback interface. In some examples the neuromodulation marker may be presented in comparison to a reference neuromodulation marker such as a difficulty threshold. For example, the neuromodulation marker measured for the subset of data at hand may be displayed next to a graphic showing a reference neuromodulation marker that is pre-determined for the subject, optionally a reference neuromodulation marker that is targeted towards a particular brain activity or therapeutic goal. Alternatively, the neuromodulation marker for the subset of data may be presented in comparison to a previously determined neuromodulation marker for a preceding subset of data. In some examples, the data is presented as a moving object, sound, or tactile stimulation (see also, Sensory Feedback, below).

By presenting the data generated during the processing phase to the subject, the system may trigger the reward system of the subject's brain. That is, the system may reward the subject if the generated data is found to be within an acceptable range or punish the subject if the generated data is outside of the acceptable range. Accordingly, in the self-regulation phase, the subject may modulate what was received during the perception phase in a therapeutic direction. Specifically, it is theorized that during the self-regulation phase, dopamine, a neurotransmitter involved in reward processing, is released by neurons in the striatum. The release of dopamine in the striatum may lead to plasticity in cortico-striatal circuits and behavioral changes (Sitaram et al. 2017, Strehl 2014). A neurofeedback interface that allows user to personalize the rewards is likely to produce better results and adherence to the training protocol. That is, the more salient the reward, the greater the release of dopamine, and the better the training outcome. Choice of the neurofeedback interface (the training environment), may be dependant on the training goal, the neuromodulation recipe, and the associated brain state.

For example, as indicated above, in some embodiments, the training environment is implemented as a video game, where different training takes place in “game worlds”. For example, the neuromodulation therapy may be directed towards cognitive enhancement and implemented as a ride or a race, with appropriately thematically designed visual and auditory feedback. In another embodiment of the system, the neuromodulation therapy may be directed towards mood enhancement training and implemented as an interactive soundscape with thematically appropriate audio voiceovers (e.g. lesson in mindfulness). In another embodiment of the system, a meditation application may be provided to the subject (e.g. lesson in calmness) either between or during neuromodulation therapy sessions.

### Neurofeedback and Neuromodulation Markers

As described above, neuromodulation markers are representations of a subset of data that are representative of brain activity that are useful for neuromodulation therapy. In accordance with one aspect of the present disclosure, a neuromodulation marker is determined using a neuromodulation recipe or function. The neuromodulation recipe, and accordingly the neuromodulation marker, may be directed towards a particular therapeutic goal.

There are multiple ways a user may might select a therapeutic goal. For example, one might want to train a characteristic that is considered sub-par or shows the highest deviation when compared to the normative data. Alternatively, a user may choose a specific characteristic to train. In some embodiments, the user may elect to proceed with a pre-set neuromodulation recipe directed towards a general therapeutic goal such as cognitive functioning or well-being.

In one embodiment, the systems and methods described herein include neuromodulation recipes that adapt to a user's brain activity and performance over time, thereby providing a personalized neuromodulation therapy that is responsive to individual conditions.

For the purpose of the discussion that follows, consider the training goal to be improving attention and the subject is to simultaneously upregulate beta power and downregulate theta power.

In one aspect of the disclosure, a reference distribution such as baseline measurements from a user or a pre-determined reference distribution, may be used to provide statistical estimates (such as percentile values), establish neuromodulation marker recipes and/or difficulty thresholds for neuromodulation therapy.

In one embodiment, a reference distribution, such as a baseline measurement, may also be used to control the difficultly level of the therapy. The level of difficulty should be set in such a way that keeps the user motivated while balancing the probability of the reward and/or punishment which is dependent on the neuromodulation marker value.

In some examples, a single baseline measurement may be obtained only once, for example, before the first training session. In other examples, a baseline measurement may be obtained before each training session. This baseline measurement may be used for the entire therapy session. It may be beneficial to update the baseline measurement of the subject prior to each session to accommodate signal quality variations due to the surrounding environment and slight variations in the placement of the headset, attentional fluctuations, drowsiness, distractions, fatigue, and other changes that commonly occur between a training sessions.

In another example, a baseline measurement may be obtained during a training session optionally at regular intervals during a session, for example, about every 15 seconds, 30 seconds, 1 minute, 90 second or 2 minutes or greater than 2 minutes. Optionally, the method or system may only adjust the baseline measurement used when determining the neuromodulation marker if a significant deviation in baseline measurements is detected or another heuristic criteria for changing the difficulty is met.

In yet another example, performance may be averaged, for example, over 60-second time intervals, at regularly spaced time offsets. If average performance drops below the 45-percentile threshold (i.e. performance has dropped below the expected value), the algorithm will re-baseline (i.e., determine a new baseline measurement to be used to determine the neuromodulation markers), effectively creating a boost, i.e. the perceived difficulty will be slightly easier than previously.

In some examples, the user may manually trigger re-baselining if the level of difficulty appears unusually low or high. In other examples, re-baselining is fully automated with an option for the user to make some adjustments.

In one embodiment, the method comprises obtaining baseline measurements for each of the individual psychophysiological parameters that make up the neuromodulation recipe. In one embodiment, the baseline measurements are then used to provide a reference distribution to determine statistical estimates of the psychophysiological parameters. In one embodiment, the method comprises obtaining baseline measurements of the neuromodulation marker. In one embodiment, the baseline measurements of the neuromodulation marker are then used to provide statistical estimates of the neuromodulation marker.

One aspect of the disclosure provides systems and methods that allow for the difficulty threshold to be varied in response to a user's motivation or skill which may fluctuate between sessions and/or within sessions. For example, if the user performs well for a while, re-baselining will set the bar higher and training will be more difficult. Following this re-baselining, if the system is not subsequently re-baselined and the user looses some motivation, the user may find that the same internal mental state that was previously rewarded, suddenly produces discouraging results.

In one aspect of the disclosure, neuromodulation markers are determined using a neuromodulation recipe. In one embodiment, a neuromodulation recipe may use a weighted ratio or sum to combine and represent the characteristics of selected psychophysiological parameters as a single value. In one embodiment, backend algorithms or machine learning may be used to determine the neuromodulation recipe, such as which psychophysiological parameters to select for the neuromodulation recipe.

As stated above, for the purposes of this portion of the description, the target goal is to increase attention span. Further, for the purposes of this portion of the description, consider that the baseline measurements indicate that the subject has unusually low beta power in focused attention tasks but fairly standard theta power.

The recipe used when training attention may be as follows:

$$R = p ( r ) , where r = {w}_{1} * p ( {sp}_{β} ) + {w}_{2} * p ( {sp}_{θ} ) ,$$

where r is the neuromodulation marker;
   w₁ and w₂ are weighting parameters;
   sp denotes spectral power of the frequency of interest (i.e., the magnitude of the amplitude); and
   p(x) denotes the percentile value of x with respect to the baseline distribution.

That is, the neuromodulation marker, when training attention, is based on two amplitude values of two psychophysiological parameters. Alternatively, the neuromodulation marker may be based on the amplitudes of three or more psychophysiological parameters.

The relative magnitude of one weighting parameter with respect to another defines the relative importance of the corresponding psychophysiological parameter in the neuromodulation recipe. The sign of the weighting parameter (i.e., positive or negative) can be selected based on the desired upregulation or downregulation of the particular psychophysiological parameter. For example, when training attention, the weighting parameter corresponding to the beta power may be positive and the weighting parameter corresponding to the theta power may be negative. As set out in Example 2, in some embodiments the weighting parameter for alpha power may be negative and the weighting parameter for theta power may be positive. In one embodiment, backend algorithms or machine learning may be used to set the relative magnitude and the sign of the weighting parameters based on training performance on previous sessions, ML derived heuristic reasons to emphasize/deemphasize one of the psychophysiological parameters, a user's change in training goals, a detection of change in user's overall cognitive-emotional state based on self-reporting or EEG analysis, or combinations thereof.

For example, the backend algorithm or machine learning may set w₁ to 4 and w₂ to −1, to emphasize beta upregulation and deemphasize theta downregulation. Further, it is to be understood that the weighting parameters may be adjusted between subsequent determinations of neuromodulation markers. For example, the subject's motivation may change throughout the session, altering the baseline measurement, which may be recognized by the backend machine learning and used to adjust the weighting parameters. In additional, the weighting parameters may be adjusted between neuromodulation therapy sessions.

When using relative magnitude values of psychophysiological parameters to determine the neuromodulation marker, it can be said that the neuromodulation markers are based on statistical estimates of the psychophysiological parameters, as opposed to raw data. For example, a psychophysiological parameter may be converted to a statistical estimate by comparing it to a reference distribution and determining a corresponding percentile value. The value of the statistical estimate of a psychophysiological parameter is therefore dependent on the raw value of the psychophysiological parameter as well as the reference distribution and is dimensionless. Similarly, the value of a statistical estimate of a neuromodulation marker is dependent on the value of the neuromodulation marker as well as a reference distribution of neuromodulation marker values and is dimensionless.

### Temporal Smoothing of Neuromodulation Markers

The human brain functions as a highly non-linear dynamical system that orchestrates a myriad of processes using finite energy resources. In self-directed neuromodulation, when a subject seeks a specific mental state in order to upregulate a given psychophysiological marker, this process is never completely definitive as to produce stable marker values. The marker value normally fluctuates when a person is not training, and may continue to fluctuate, even during a concerted effort to bring the marker towards a specific range. Accordingly, it is preferable that feedback to the user, such as in the form of visual or auditory representations of marker data, be smoothed, otherwise, it may appear perceptually confusing and even unpleasant.

In the field of neurofeedback, it is generally considered desirable to have shorter delays, closer to real-time feedback, while data smoothing results in a slower response and more lagging. Accordingly, it is important to carefully balance between these competing requirements in order to provide useful feedback for neuromodulation training.

In one embodiment, the system and methods described herein comprise temporally smoothing the feedback provided to a subject such as by temporally smoothing the neuromodulation marker.

In some embodiments, the temporal smoothing is based on a moving weighted average with a window length of between 0.2 and 2 seconds, optionally between 0.5 and 1.5 seconds or about 1 second. In some embodiments, the temporal smoothing is based on an overlap between consecutive windows of between 25 ms and 1 second, optionally between about 100 ms and 500 ms or about 250 ms between consecutive windows. In a preferred embodiment, the temporal smoothing is based on a moving weighted average with a window length of between 0.5 and 2 seconds, optionally about 1 second, and based on an overlap between consecutive windows of between 50 and 500 ms, optionally about 250 ms between consecutive windows.

In one embodiment, temporally smoothed neuromodulation marker Rₛₘₒₒₜₕ is determined using the formula:

$${R}_{smooth} = {\sum }_{i = 1}^{4} \frac{i}{{\sum }_{j = 1}^{4} j} \cdot R ( i )$$

where R(i), i=1 . . . 4 represents 1 second history of R-values.

In some embodiments where R is obtained by averaging percentile values. Its theoretical range is 0-100. Rₛₘₒₒₜₕ may be the final output of the computing device and it is this value that may be, when using a system that implements smoothing of neuromodulation markers, translated into sensory cue(s), and presented to the subject.

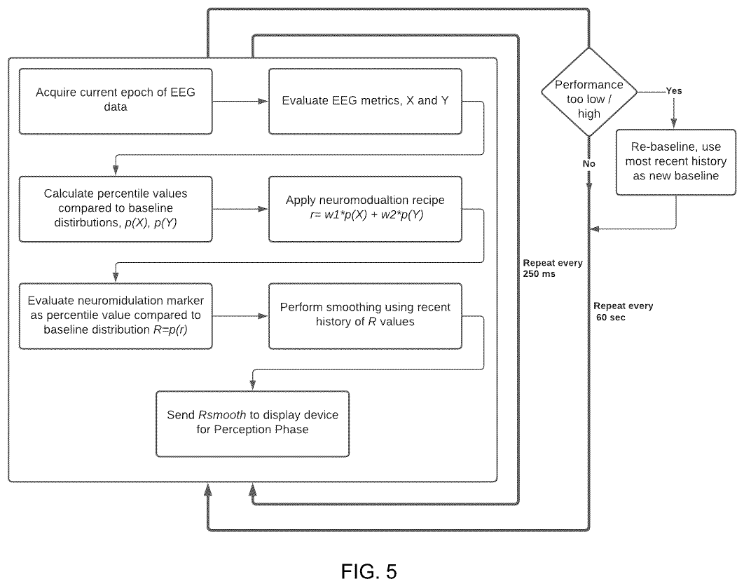

An exemplary scheme that uses a smoothed neuromodulation marker Rₛₘₒₒₜₕ to provide feedback to a user is shown in FIG. 5.

### Advantages of Neuromodulation Recipes

Using a neuromodulation recipe that uses statistical estimates such as percentile values to determine the neuromodulation marker improves the variability and smoothness of the final estimate R, making the feedback more perceptually congruent. That is, the feedback is more user friendly when using neuromodulation recipes as described above because the system uses percentile values at one level, and optionally two levels: (a) in the neuromodulation inputs (e.g. p(sp_{β}), p(sp_{θ})) (and optionally (b) the neuromodulation marker output (e.g. R=p(r)). Furthermore, the use of neuromodulation recipes with weighting parameters for each psychophysiological parameter allows for neuromodulation therapy that is responsive to individual differences and training goals and is effective across a diverse population of users. As shown in Example 2, neuromodulation therapy that used weighted parameters and percentile values at two levels was effective at improving quantitative measures of wellness in test subjects.

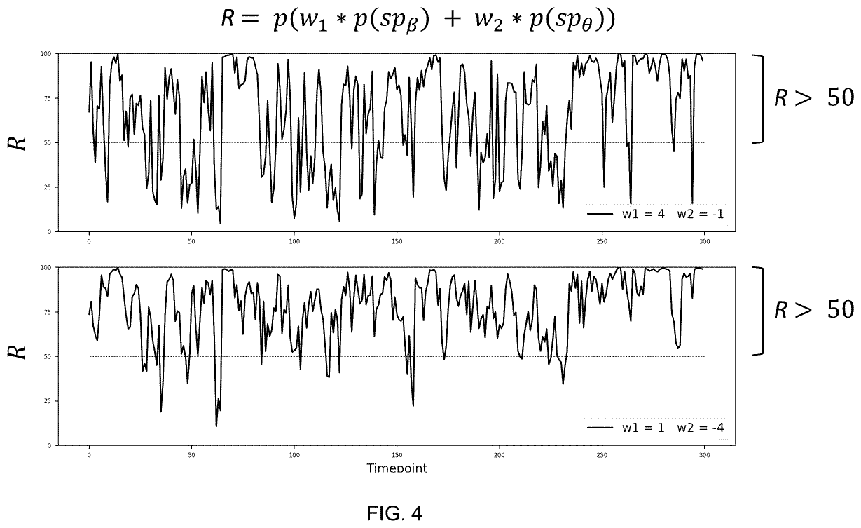

FIG. 4 shows two examples of r-values obtained with different weights. Assuming a simplified scenario where the user receives positive feedback when R>50, note how the frequency of positive feedback differs between the two recipes. Replacing raw values with percentiles may have a major advantage in that it equalizes components in terms of their respective contributions to the overall variance while allowing the weights to precisely define those contributions regardless of the underlying raw values and their variability. In other words, percentile values may allow for a recipe to universally capture therapeutic goals regardless of within-subject and across-subject variations of absolute measures (biological variability).

It is to be understood that the above formula showing how to determine neuromodulation markers does not limit the system and method described herein to the use of EEG sensors or to relative power in theta and beta frequency ranges. Indeed, neuromodulation recipes may use other brain imaging modalities or other EEG metrics as component psychophysiological parameters.

Further, standard EEG metrics other than relative spectral power, such as global field power (GFP), absolute spectral power, entropy, and coherence or asymmetry between different electrode sites, may be used a psychophysiological parameters. Further yet, the use of statistical measures allows a recipe to combine completely disparate metrics. For example, the recipe:

$$r = 3 * p ( {sp}_{β} ) - 1 * p ( GFP )$$

prescribes simultaneous upregulation of relative beta power and downregulation of GFP, with beta power being 3 times more important.

By using recipes based on statistical rather than raw values, sources of variability such as biological, environmental, and hardware may be minimalized (i.e., may have a reduced effect on the subjects ability be rewarded by the system).

For example, interference from electrical equipment (60 Hz in North America) can vary a lot, depending on the surrounding environment, e.g. park bench vs computer lab, kitchen vs bedroom, etc. When using a neuromodulation recipe to determine the neuromodulation marker this interference may be reflected in the baseline measurements, and the neuromodulation marker can be adjusted accordingly.

### Sensory Feedback

Neuromodulation therapy is intended to be a non-invasive method of changing the brain by helping user increase their natural abilities. To do so, as described above, the system may provide at least one form of sensory feedback to the subject. In one embodiment, the sensory feedback may include manipulation of an object on a screen (i.e., moving, resizing, changing color, transparency, blur, and/or adding visual effects). In another embodiment, the sensor feedback may include the use of auditory cues, such as playing music and/or sound effects.

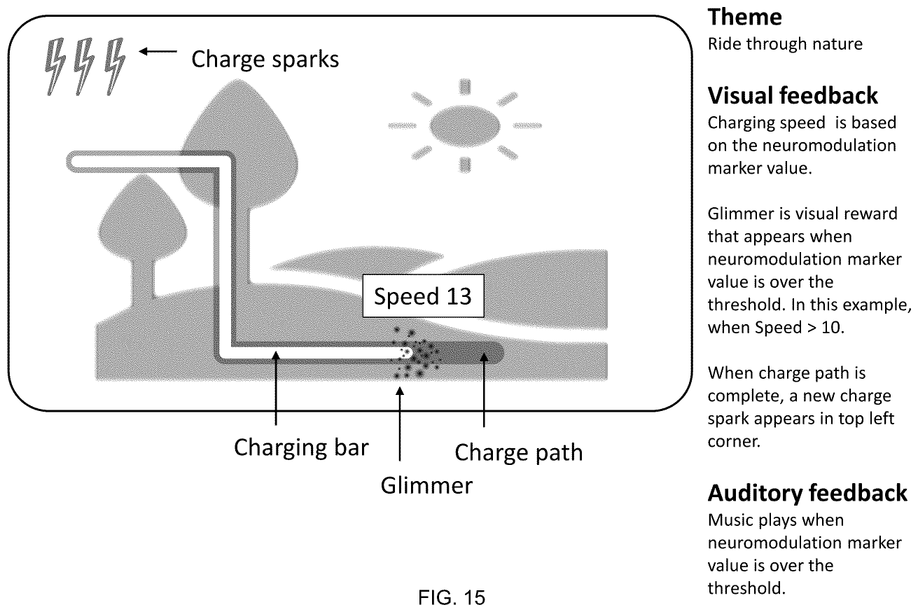

Alternatively, cross-modal cues may be used which may lead to enhanced speed and accuracy of detection of objects and events, and the choice of appropriate responses (see for example Evans, 2010; Patching, 2004). That is, the system may use cross-modal combination of visual and auditory cues. Cross-modal cues may be presented to the subject via a single output device or by multiple output devices. An example of a user interface for presenting sensory feedback to a subject is shown in FIG. 15. In one embodiment, the methods and systems described herein provide perceptually congruent feedback by using mathematically different cueing for each sensory modality.

In one embodiment, the sensory feedback may use tactile stimuli with or without cross-modal combinations with visual and auditory modalities.

In one embodiment of the system, visual feedback is obtained by directly converting a neuromodulation marker such as Rₛₘₒₒₜₕ (see, Data Smoothing, above) into movement speed of an object on the screen and the subject may be instructed/motivated to increase speed. In the same embodiment, auditory cuing may also be given, such that music is played as positive feedback and it fades out to indicate the wrong direction. Because rapid fluctuations may be considered jarring, the music volume may be treated differently. The basic premise for the user is to perceive music as a positive reward, so the volume expresses consistent movement towards or away from the target state according to the following iterative temporal smoothing formula.

For example, in one embodiment, L is a number between 0 and 100 which determines the level of difficulty. A higher value of L implies harder training. Default is 50. Let nct and pct denote negative and positive counter thresholds that will determine temporal smoothing for music volume. Assuming that iterations are performed every 250 ms, default values of are nct=−1, pct=1.

```
START count = 0
FOR EACH ITERATION
  $d = \{ \begin{matrix}\frac{{R}_{smooth} - L}{L} , {R}_{smooth} \leq L \\ \frac{{R}_{smooth} - L}{100 - L} , {R}_{smooth} > L\end{matrix}$
    count = count + d
IF count > 0 THEN
  music volume = 1
IF count > pct THEN count = pct
ELSE
  IF count < nct THEN count = nct
  music volume = 1 − count /nct
```

In one embodiment, parameters of sensory feedback are varied over time in response to user input, analysis of user data or a combination thereof. In one embodiment, algorithms or optionally backend machine learning (ML) is used to find optimal settings for each user.

### Method for Providing Neuromodulation Therapy

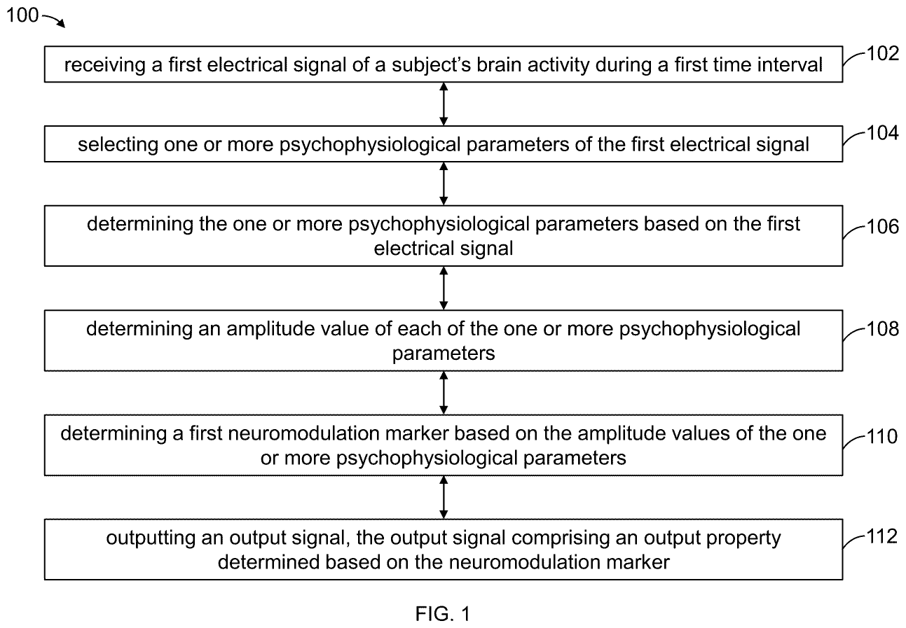

Reference is now made to FIG. 1, in which a method 100 for providing neuromodulation therapy to a subject is outlined. Method 100 is an example method of providing neuromodulation therapy in accordance with the description above. Accordingly, the examples discussed below may be applied to the discussion above and the discussion above can be applied to the examples discussed below.

Method 100 begins at step 102 with receiving a first electrical signal of a subject's brain activity during a first time interval. A processor, in communication with a sensor able to obtain electrical signals of the subject's brain activity, may be used at step 102 to receive the first electrical signal.

Any sensor known in the art may be used to obtain the electrical signal of the subject's brain activity. As described above, the electrical signal may be obtained using any one of, but not limited to, EEG, fMRI, MEG, and fNIRS. The sensor can be implemented as a headset that wirelessly connects to a computing device. Specifically, in one embodiment the sensor may be any one of, but not limited to, EEG headsets, such as MindWave Mobile by Neurosky, Muse by Interaxon, Insight or Epoc by Emotiv. In one embodiment, the sensor is an EEG with a sampling rate of 128 Hz or higher. In one embodiment, the sensor is configured for wireless data transmission via Bluetooth or other protocols for the wireless transmission of data. In some embodiments, the system can include, in addition to a sensor for generating an electrical signal of brain activity, an accelerometer and/or other biosensors such as an electrocardiogram (EKG), electromyogram (EMG), or Galvanic skin response (GSR). In one embodiment, the method further comprises positioning the sensor, such as an EEG sensor, on the subject prior to measuring the electrical signal of the subject's brain activity.

At step 104 of method 100, the processor selects one or more psychophysical parameters of the first electrical signal. For example, if the first electrical signal is EEG data (such as, for example, voltage data and optional multichannel voltage data) that is transformable into Fourier domain measurements of spectral power within known frequency bands, for example, delta (1-4 Hz), theta (4-7 Hz), alpha (8-12 Hz), beta (15-30 Hz), and gamma (35-45 Hz), the processor may select a single frequency band (e.g. theta), two frequency bands (e.g. beta and theta), or three or more frequency bands as the psychophysical parameters. As described above, the selection of the one or more psychophysical parameters of the first electrical signal may be based on user input and/or subject data (i.e., by an algorithm using stored data) or default to one or more presets. The selection of the one or more psychophysical parameters of the first electrical signal may advantageously enable the determination of neuromodulation markers for use in BCI technologies based on a subset of sensor data received from the sensor, and thus decrease the processing overhead.

The processor selects one or more psychophysical parameters of the first electrical signal at step 104 because, depending on the type neuromodulation therapy to be provided, all of the psychophysical parameters within the first electrical signal may not be required. That is, to provide neuromodulation therapy, in some examples, only data pertaining to certain frequencies may be required (e.g. theta band); in other examples, data pertaining to a plurality of frequencies may be required (e.g. each of delta, theta, and beta). The sensor data including, for example, each of delta, theta, and beta frequency bands may be a subset of the data received from the sensor, and may therefore advantageously improve the functioning of the processor by reducing overhead.

At step 106 of method 100, the processor determines the one or more psychophysiological parameters based on the first electrical signal. For example, if alpha frequencies were selected as a psychophysiological parameter in step 104, the processor may determine, i.e. isolate, the alpha frequencies that are present within the first electrical signal. Put another way, the first electrical signal may contain data that represents more than just the selected psychophysiological parameters, and therefore the data representing the psychophysiological parameters must be separated, i.e., determined from, the first electrical signal.

At step 108 of method 100, the processor determines an amplitude value of each of the one or more psychophysiological parameters. The amplitude value of each of the one or more psychophysiological parameters is required to characterize the measured psychophysiological parameters. That is, in one embodiment brain activity may be evaluated based on the amplitudes of the one or more psychophysiological parameters, such as the amplitudes of brainwaves at certain frequencies, and used to provide feedback to the subject.

At step 110 of method 100, the processor determines a first neuromodulation marker based on the amplitude values of the one or more psychophysiological parameters. In some examples, the neuromodulation marker is determined using a neuromodulation recipe, as described in detail above. In some examples, the neuromodulation marker is determined based on statistical estimates of the amplitudes of the one or more psychophysiological parameters. In some embodiments, the first neuromodulation marker is a statistical estimate such as a percentile relative to a distribution of neuromodulation marker values for a particular neuromodulation recipe, optionally a baseline distribution of neuromodulation marker values obtained from the subject at the start of the therapeutic session. The use of statistical estimate instead of raw amplitude may advantageously make the determination of neuromodulation markers for use in BCI technologies more robust. For example, any constant presence of electrical interference from the surrounding environment (e.g. generated by nearby home appliances computers etc) may greatly impact raw amplitudes of target frequency bands, but not in a statistical estimates. The use of statistical estimates emphasizes relative changes compared to a reference distribution, no matter how much noise may be present in the underlying signal, as long as it relatively constant during over the course of a single session.

At step 112 of method 100 an output signal comprising a first output property based on the first neuromodulation marker is outputted to an output device in communication with the processor. The output device may be at least one of, but not limited to, a display device (such as, for example, a mobile phone, laptop, a VR or AR headset, or projection space), an audio output device, and a tactile device. Accordingly, the output signal may be any one of, but not limited to, a visual output displayed on the display device, an audio output emitted from the audio output device, and a tactile output generated by the tactile device.

In some embodiments, processing and display functionalities (i.e., the output device) are provided by a single device such as a mobile phone, tablet, laptop, or desktop workstation. In some embodiments, data processing and display are performed in near-real-time, with refresh rate ranging between 5 and 500 ms.

As stated above, the first output property is based on the first neuromodulation marker. That is, the first output property is a representation of the first neuromodulation marker. In one embodiment, the first neuromodulation marker is a statistical estimate of the neuromodulation marker relative to a reference distribution, optionally a percentile value relative to reference distribution of the subject's own data. The first output property may be in any format that allows for feedback regarding brain activity and therefore neuromodulation therapy to be provided to the subject.

In some examples of method 100, the method may further comprise receiving a second electrical signal of the subject's brain activity during a second time interval. The second time interval may be immediately following the first time interval or may be separated from the first time interval by a period of time.

Similar to the first electrical signal, the processor may (a) select one or more psychophysiological parameters of the second electrical signal; (b) determine the one or more psychophysiological parameters based on the second electrical signal; (c) determine an amplitude value of each of the one or more psychophysiological parameters; (d) determine a second neuromodulation marker based on one or more amplitude values of the one or more psychophysiological parameters. In some embodiments, method 100 comprises providing feedback to the subject, at the output device in communication with the processor, by changing the output signal to a second output property determined based on the second neuromodulation marker. In some embodiments, method 100 comprises providing feedback to the subject based on temporally smoothing at least at a first neuromodulation marker and a second neuromodulation marker. For example, step 112 may involve outputting an output signal, the output signal comprising a first output property determined based on temporally smoothed neuromodulation marker data for the subject.

In some embodiments, method 100 comprises providing feedback to the subject out the output device based on at least one neuromodulation marker relative to a difficulty threshold.

In some examples, the one or more psychophysiological parameters of the first electrical signal are the same type as the one or more psychophysiological parameters of the second electrical signal. This may be the case during a training session or between multiple training sessions, optionally wherein the neuromodulation recipe remains the same. Alternatively, the one or more psychophysiological parameters used to determine the neuromodulation marker and provide feedback to the subject may change over time, either during a training session or between training sessions.

In some examples, when the one or more psychophysiological parameters of the first electrical signal are the same type as that of the second electrical signal, the output signal provided as feedback may be determined based on the difference between the first neuromodulation marker and the second neuromodulation marker. In some embodiments, the output signal provided as feedback may be presented relative to a difficulty threshold.

In some examples of method 100, the processor may be in communication with a form of memory that includes a subject database. The subject database may include subject data, such as, but not limited to, electrical signal data, psychophysiological parameter data, amplitude value data, neuromodulator marker data, subject testing data, and/or subject annotation data. The subject database may include reference distributions for one or more psychophysiological parameters or reference distributions for one or more neuromodulation markers.

In some examples of method 100, subject data may be transmitted from a network device in communication with the processor to a remote server. The subject data may include, for example, at least one selected from the group of electrical signal data, psychophysiological parameter data, amplitude value data, neuromodulator marker data, subject testing data or subject annotation data.

The method 100 may also include receiving, at the network device from the remote server, at least one selected from the group of a revised difficulty threshold and one or more adjusted weighting parameters. Accordingly, within the method 100, the difficulty threshold or weighting parameters may be adjusted based on the revised difficulty threshold or adjusted weighting parameters, respectively.

### System for Providing Neuromodulation Therapy

In another aspect, there is provided a system for providing neuromodulation therapy to a subject. In one embodiment, the system is configured for providing neuromodulation therapy according to a method as described herein. In one embodiment, the system comprises at least one sensor, a memory, a processor in communication with the at least one sensor and the memory, and an output device for providing feedback to a subject, such as an output signal comprising an output property. In one embodiment, the processor is configured to perform all of the functions outlined herein. Alternatively, the processor may be operably connected to a remote server which performs one or more of the functions outlined herein.

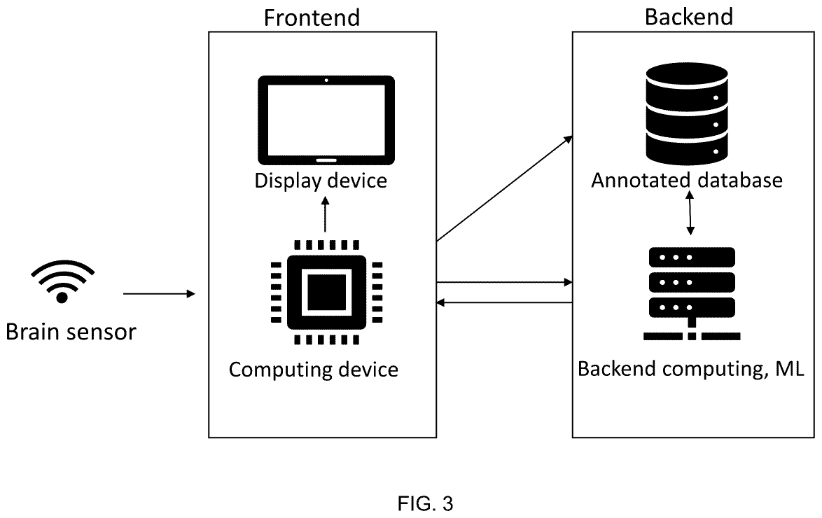

Referring now to FIG. 3, shown therein is an example system for providing neuromodulation therapy. In the example illustrated the system includes a brain sensor such as an EEG, which passively monitors the user's brain activity; a computing device (i.e., a processor and memory); and a display device, which may or may not be combined with the computing device into a single unit.

The computing device may process, for example, the raw EEG data and derive a specific neuromodulation marker using a neuromodulation recipe. The amplitude of the neuromodulation marker in the form of a reward or punishment may momentarily be communicated to the subject via visual, auditory, or tactile output on the display device, or within a virtual environment. In doing so, the system implements a closed-loop regime such that the subject simultaneously perceives and modulates their ongoing brain activity. As demonstrated in Example 1, repeated use of the system may facilitate long-term strengthening of cognitive function. The system may also incorporate remote data storage and backend machine learning algorithms for adaptive personalization of neuromodulation recipes based on data inputs across cumulative user interactions with the system over time.

### Combined Training

In some embodiments of the method or system, multiple characteristics of the subject may be trained at the same time. For example, neuromodulation therapy for creativity may be combined with memory training with infrequent popup tasks that require a short attentional shift away from the ongoing creativity training. Other types of cognitive training and activities such as breathing exercises, creativity exercises, physical exercise can also be implemented. As shown in Example 2, the method and system described herein have been demonstrated to be useful for training multiple psychophysiological parameters using a neuromodulation recipe that combines weighted parameters into a single neuromodulation marker.

In another embodiment of the present method or system, elements of talk-therapy, mindfulness, hypnosis, and positive psychology may be incorporated. For example, so-called “alpha-theta” training is often used in clinical practice for treating PTSD and addictions. The essence of such training is to first create intentions, then proceed with neurofeedback training with alpha-theta or similar protocol that induces hypnagogic state. In this state, the critical mind is temporarily deactivated, and the person is more receptive to suggestions.

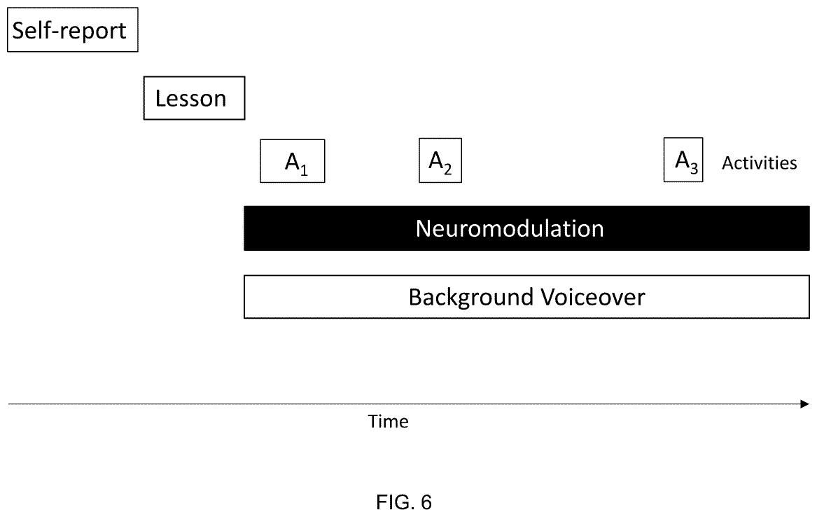

In one embodiment the present method or system implements a training program for cognitive-behavioral modification consisting of a series of training sessions, such that each session starts with a short audio or video lesson, followed by an neuromodulation training session targeting relaxed or suggestable brain state, with voiceovers playing in the background. Lesson and voiceovers may be created by a personal therapist, the user themselves, downloaded from the internet, or any combination thereof. The modular structure of training sessions is illustrated in FIG. 6.

One embodiment of the present method or system may be used in psychotherapy such that the therapist can prescribe a customized training program by selecting a combination of lessons, cognitive tasks, voiceovers and neuromodulation recipes. Another embodiment of the present system can be used in life or performance coaching such that a coach can prescribe a customized training program by selecting a combination of lessons, cognitive tasks, voiceovers and neuromodulation recipes.

### Annotated Brain-Behavior Database

The method and system as described herein may also be set-up to track brain activity under different scenarios. These scenarios may include prescribed activities such as resting state, cognitive tests, retrospection, self-evaluation, relaxation exercises, breathing exercise, watching or listening to different content, etc. Along with brain activity, the system may also record annotations so that brain data can be precisely timed and analyzed in relation to the user's interactions with the system. That is, the system may generate an annotated brain-behaviour database. For example, an annotation may record user's gestures on the touch screen, responses to questionnaires, events triggered by the system, such as popup instructions, etc.

It may be desirable to compile an annotated brain-behavior database as it allows the system to track longitudinal data for each individual over time. Repeated scenarios may be of particular interest as they can be used to understand longitudinal changes of brain and behavior. The database also may be used to cross-sectionally compare training trajectories between individuals and matching groups, where matching may be based on demographic factors such as age and education. Both longitudinal and cross-sectional data may be used to detect significant deviations from the norm and may be used for diagnostic purposes.

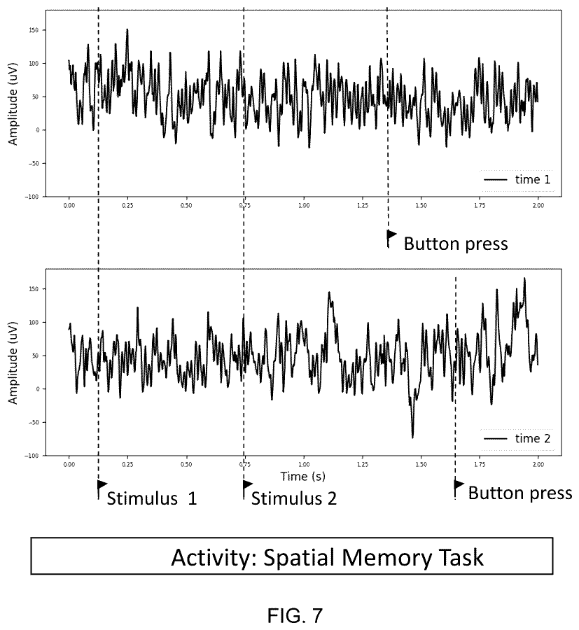

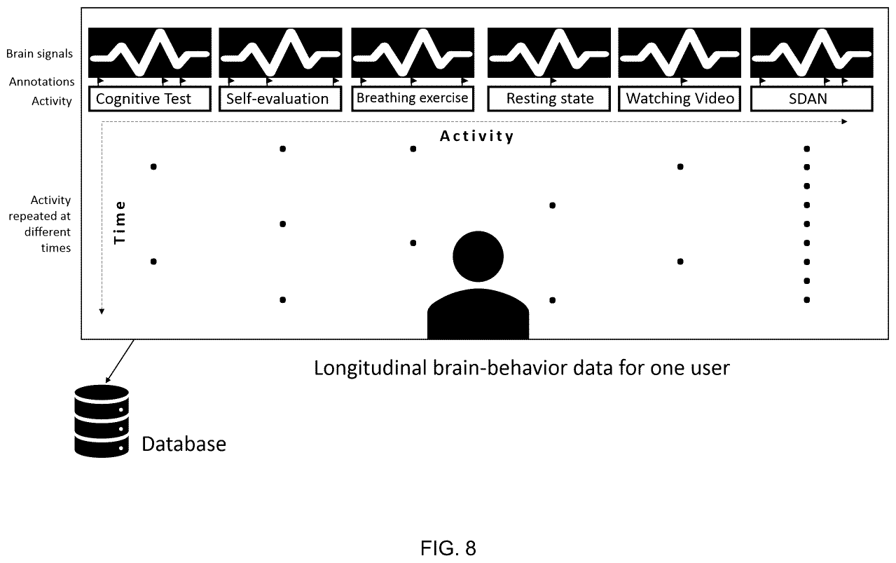

FIG. 7 illustrates an example of annotated brain-behavior for one subject and the same activity, Spatial Memory Task, performed at two different time points. A schematic example of an annotated database is shown in FIG. 8.

### Backend Data Analysis and Personalized Training Trajectory

The method and system as described herein may also be set-up to allow for a personalized training trajectory. The system may start by acquiring an initial personal snapshot. This may include identifying the user's goals and concerns. For example, the user may want to improve their cognitive performance. Based on this information, the system may determine an initial neuromodulation recipe. The snapshot may also include a battery of neurocognitive tests and self-reports. The snapshot may be acquired in one go (i.e., in one training session), or it may be broken down into smaller components and distributed across several training sessions.

Based on the brain activity data and performance metrics from the snapshot, the system may adjust the neuromodulation recipe. For example, one type of adjustment is the re-definition of frequency bands according to individual peak alpha frequency (PAF). PAF may be determined from resting-state EEG during eyes open and eyes closed conditions. Individualized frequency bands may be defined at fixed distances from PAF (Corcoran, 2018).

Another recipe adaptation may be based on a comparative analysis of EEG data with a normative database containing reference data. An example of such adaptation is to include beta power downregulation if it is determined that an individual has unusually high beta levels. Additionally, changes in wellbeing and performance metrics may trigger recipe adaptation or switch in the training trajectory. For example, if unusually high levels of stress are detected, the system may either adjust the neuromodulation recipe to include beta downregulation or switch the training to pure relaxation.

Another aspect of the present system may be in applying machine learning (ML) to the annotated brain-behavior database. For example, machine learning may identify principal training profiles by applying dimensionality reduction and clustering algorithms. Furthermore, machine learning may use predictive modeling to forecast training outcomes and use this information to further optimize the training trajectory for each user.

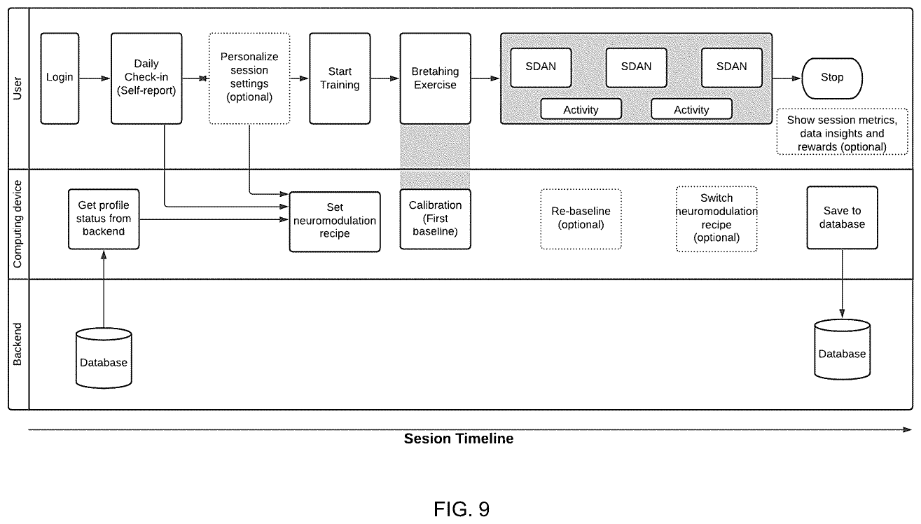

An example timeline of a single neuromodulation training session is shown in FIG. 9.

### Tracking of Transfer Effects

In one embodiment, the method and system described herein comprise providing a testing application to the subject using the processor and the output device and storing subject testing data in a subject database. Optionally, the subject database is a remote database operatively connected to the processor through a network device. In one embodiment, the testing application is a cognitive testing application.

In the present method and system, the interface and user experience of the tests may be markedly different and independent from the neuromodulation exercises and thus the system may avoid the pitfalls of “teaching to the test”, a common problem with interventions that measure cognitive gains by using the test that is similar to the training itself.

Accordingly, transfer effects measured in this way may be considered to be desirable. For example, if a user selects cognitive performance at their goal, the system may design neuromodulation exercises for the prefrontal cortex to increase cognitive capacities such as memory and attention. In order to quantify the transfer effects, the system may incorporate a battery of independent tests. These tests may be taken before the training begins and at scheduled intervals. For example, the tests may be performed on every 10, 15, 20 or 25 sessions or upon reaching training milestones. Alternatively, the test may be incorporated into a game with different popup tests on every session, until the full battery of tests is administered, then re-taking the tests in the same order in the subsequent sessions.

In one embodiment, those same tests are repeated infrequently, so that tests themselves are not driving the changes. Formal and informal beta-testing of different embodiments of the current method and system suggested that a preferred frequency for repeating cognitive testing for transfer effects is after about every 6 hours of active training, or approximately once a month, assuming regular daily training schedule. In some embodiments, the method and system described herein comprises testing for transfer effects after at least about 5 hours, 6 hours or 7 hours or active training, optionally between about 5 hours and 7 hours of active training.

In some embodiments, the method and system may implement cognitive tests such as the ANT, NBACK and/or FAS tests described in Example 1. In some embodiments, the method and system may implement Trail Making Test (TMT), a standard and well-studied test of executive function. TMT's genetic aetiology has already been identified and also correlated with the generalized cognitive performance (Hagenaars et al., 2018). The method and system may also include the Stroop test, both standard and emotional versions, which measure cognitive control, selective attention, impulsivity, and processing speed (ability to quickly make decisions while inhibiting interference from irrelevant information). The main dependent measure in the Stroop task is the “Stroop Effect”, the degree of slowing, and the reduction in accuracy for incongruent relative to congruent trials. The Stroop Test has shown sensitivity to age, stress and mood (Ando, 2017; Zurrón, 2014). The system may also implement visuomotor Reaction Time Test (RTT). RTT can predict athletic performance (Eckner, 2011), impulsivity, cognitive decline or neurodegenerative processes (Kochan, 2017). The method and system may also include Corsi-block tapping Test of visuospatial short-term memory and N-Back test for working memory. The system may also include Alternating Finger Tapping Test for measuring motor control (speed, accuracy, spatial and temporal precision) and Standing Balance Test.

### Self-Assessments

In one embodiment, the method and system described herein further comprise providing a self-evaluation application to the subject using the processor and the output device and storing subject self-evaluation data in a subject database.

For example, the present method and system may include self-assessment tools for tracking psychological metrics of wellbeing such as sleep, mood, emotional wellbeing, productivity, etc. These tools may be implemented as multiple-choice questions, level sliders, and other common online survey formats.

Formal and informal beta-testing of different embodiments of the current method and system suggested that a preferred frequency for administering self-assessments is every session, at least every second session or at least every third session, at least every 4 sessions or at least every 5 sessions.

### Data Insights

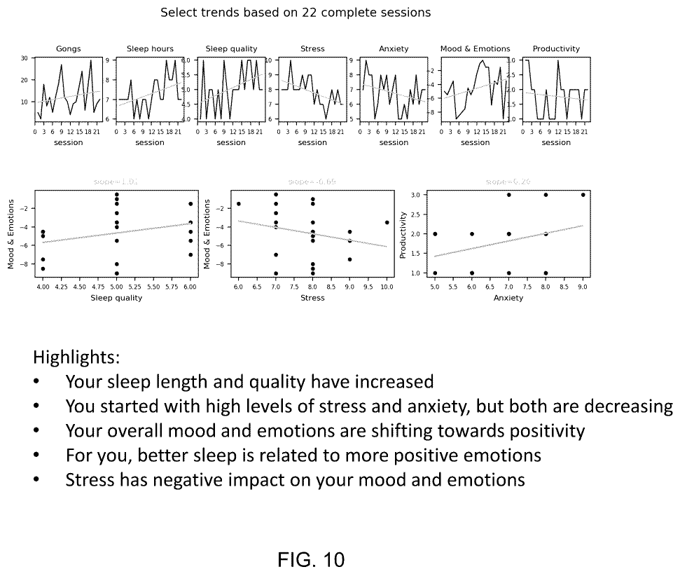

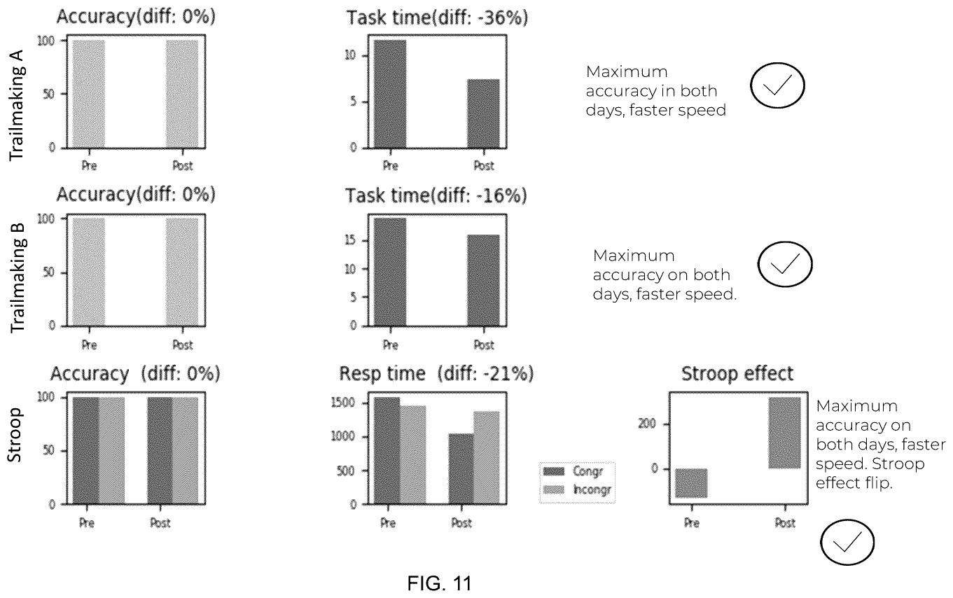

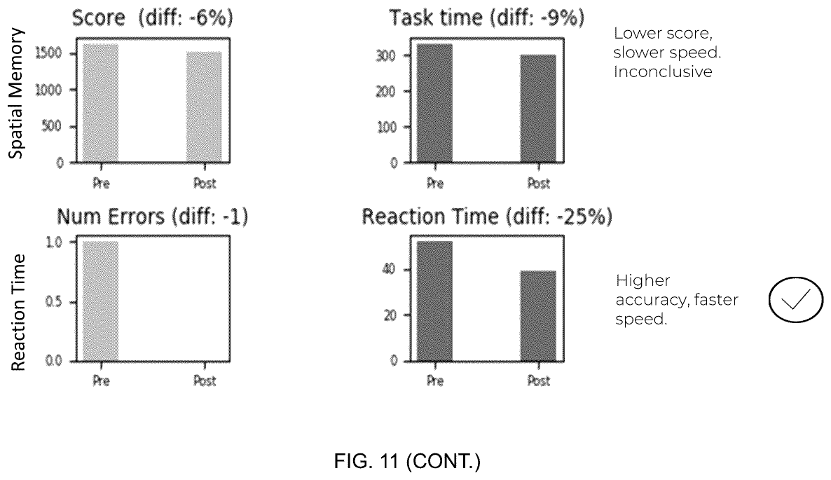

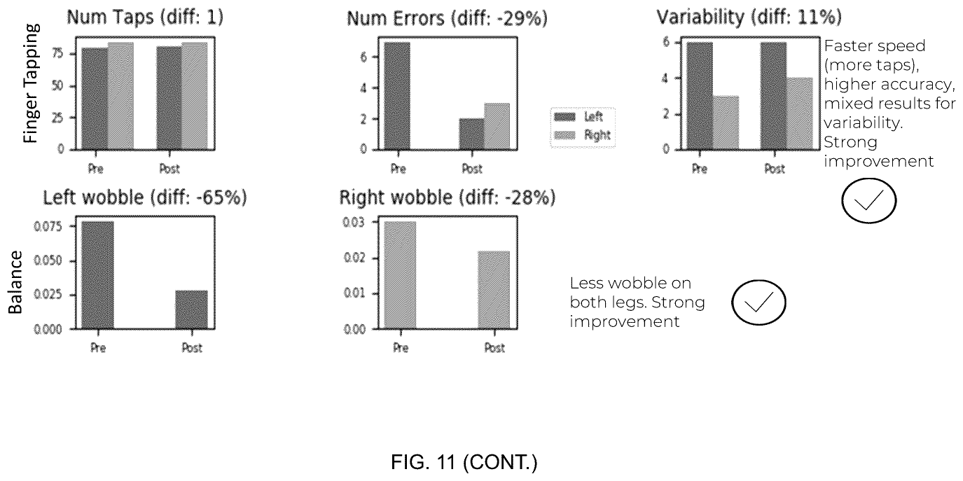

The present method and system may implement data reporting for users. These data reports or insights may contain numeric and graphical representations of various data slices from the database. These may include neuromodulation metrics, rewards, quantified transfer effects, trends in wellbeing metrics, and correlations among these different data sources. The reports may also include comparisons with the results of other users or groups, which may or may not be matched in terms of demographic factors. An example of a report of trends in wellbeing metrics is shown in FIG. 10. An example report of transfer effects is shown in FIG. 11.

### Dashboards

The present method and system as described above may implement online dashboards for data visualization, interpretation, and management. The system may implement multiple roles for different types of access, with specific viewing and editing privileges. For example, an ‘admin’ role may be given the highest access privileges. Another example role is that of a ‘coach’. A coach may be able to add new users, manually adjust or override a subset of training parameters, and visualize individual and aggregate user data. A coach's dashboard may also include summary views of longitudinal and cross-sectional analyses of user's data. Furthermore, a ‘Data analyst's role may allow raw data downloads and processed data uploads. The system may use automated push notifications to communicate significant data events to multiple users of the system, in accordance with their roles. In doing so, the system may support and synchronizes coach-client relationships and allow dynamic integration of the scientific research and machine learning.

### EXAMPLES

### Example 1: Enhancing Cognitive Performance in Older Adults With Mobile Neurofeedback Training

A study was conducted to in order to assess the use of neuromodulation therapy with feedback generated based on statistical estimates (percentile values) of EEG activity and to evaluate transfer effects in a population of older adults.

### Experimental Design and Protocols

### Test Subjects

80 older adults with normal cognition were recruited for the experiment. Pre-screening for normal cognition was performed using Mini-mental state examination (MMSE, score>27), Montreal Cognitive Assessment (MOCA, score>25) and Back's Depression Inventory Index (BDI-II, score<14).

### EEG Data Acquisition

Electroencephalographic (EEG) activity was recorded using BrainLink EC wearable headset with Neurosky's TGAM chip. EEG was recorded at a sampling rate of 512 Hz. The headset captures single-channel raw EEG data as the potential difference between the electrode placed on the forehead and the reference electrode placed on the left earlobe.

### Control for Placebo Effects

Participants were randomly assigned to one of the two groups: intervention group (Training Neurofeedback, TNF) or active control group (Randomised Neurofeedback, RNF). Participants from both groups received neuromodulation training with an identical user interface, except that TNF group consistently trained to upregulate theta power in all sessions, while participants from the RNF group received a randomized target frequency band in each session. Frequency bands used for randomization were: 4-8 Hz (theta), 8-12 Hz (alpha), 13-15 Hz (beta1), 8-30 Hz (beta2) and 35-50 Hz (gamma). Randomization also included a choice between upregulation (+) and downregulation (−). For the RNF group, the algorithm randomly chose between one of theta−, alpha−, alpha+, beta1−, beta1+, beta2−, beta2+, gamma−, gamma+. In this way, the control group received true neurofeedback on each session, but training effects were not reinforced across sessions. Both participants and staff were blinded about group membership.

### EEG Processing for Neurofeedback

Self-directed active neuromodulation (SDAN) was implemented using a proprietary mobile application, Memoride which ran on an iPad and received raw EEG data from the headset over Bluetooth. Incoming raw EEG was segmented into 1-second epochs of continuous data. Epochs with maximum absolute amplitude surpassing 200 uV were deemed noisy and were rejected. Clean epochs were decomposed using Fast-Fourier Transform with a frequency resolution of 1 Hz. 5 frequency bands of interest were defined and selected: 4-8 Hz (theta), 8-12 Hz (alpha), 13-15 Hz (beta1), 8-30 Hz (beta2) and 35-50 Hz (gamma). For each frequency band, absolute band spectral power was calculated as a sum of squared magnitudes of the Fourier coefficients at frequencies within the said band. Relative band spectral power was calculated as band absolute spectral power divided by the absolute power of 1-50 Hz range. Spectral power calculations were repeated every 250 ms.

The training started with a paced breathing exercise, which was also used as calibration. During this time, the screen displayed a rotating circle and instructions for a three-part breathing cycle, inhale-hold-exhale, each part lasting 4 seconds, for a total of 12 seconds for the full cycle. Calibration lasted 1 minute or more until 240 clean epochs were acquired. This data was taken as a baseline for subsequent neuromodulation. Immediately following the calibration, the neuromodulation training commenced. Memoride was themed as a virtual bike ride with bike speed as a visual cue and music volume as the auditory cue. The cues were triggered by the relative power of the target frequency band. For each new EEG epoch (every 250 ms), relative spectral power of the target frequency was expressed as a percentile value compared to the baseline, resulting in a number between 0 and 100, denoted R.

This R value, after temporal smoothing (see below), was converted into the bike speed. Whenever Rₛₘₒₒₜₕ was greater than 50, the user was rewarded with red flames behind the bike and audible music. This suprathreshold speed range was communicated to the user as “being in the zone”. When Rₛₘₒₒₜₕ was less than 50, the flames and music faded. The bike moved in a straight line from the left to the right edge of the screen. Each full ride across the screen was considered a lap. The training session consisted of 3 riding blocks of 5 minutes each. On average, there were 14 laps within each 5-minute block. At the end of each block, the user was presented with a memory recall task (see below).

### Smoothing and Visual and Auditory Feedback

Spectral power of the target frequency was temporally smoothed using a moving weighted averaging procedure with window length of 1 second with 250 ms overlap:

$${R}_{smooth} = {\sum }_{i = 1}^{4} \frac{i}{{\sum }_{j = 1}^{4} j} \cdot R ( i )$$

Where R(i), i=1 . . . 4 represents percentile values of relative spectral power of the target frequency band over the last second.

The visual interface in Memoride animated the bike movement according to rₛₘₒₒₜₕ and displayed this value converted to a naturalistic speed by multiplying it by 0.2. For example, the maximum percentile value of 100 was mapped to the maximum bike speed of 20 km/h and threshold value of 50 was mapped to 10 km/h. In other words, the neuromodulation marker was translated into bike speed and the user was instructed to ‘ride’ as fast as possible. Ride speed over 10 km/h was rewarded with visual and auditory effects. Average speed, top speed, percent time in the top speed zone, are examples of user-facing metrics that were derived from the neuromodulation marker.

Memoride also monitored the user's ability to sustain a Rₛₘₒₒₜₕ value above 50. The longest continuous time interval with Rₛₘₒₒₜₕ>50 during a session was termed the “longest hold”. Tracking this value within and across training sessions captures another aspect of neurofeedback learning.

### In-training Memory Tasks

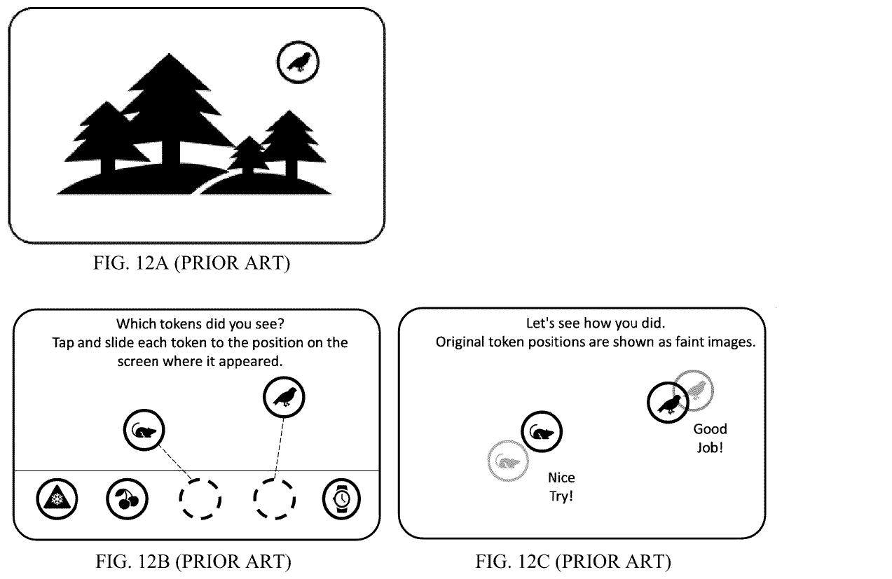

In addition to SDAN training Memoride incorporated a proprietary spatial memory task. During each 5-minute training block, a round token occasionally popped up on the screen. The user then had 3 seconds to collect the token by tapping it and was required to remember the image and position on the screen (see FIG. 12.A). The frequency of token popups was proportional to the ride speed. In other words, better SDAN performance resulted in higher speed and more frequent testing of higher working memory load. The number of tokens ranged between 0 and 5, where 5 could only be attained if the ride was at the top speed for the entire 5 minutes. Most of the time, the number of popup tokens was 1 to 4 per 5-minute block. At the end of the training block, the user was presented with a row of tokens at the bottom of the screen including all tokens that appeared plus two more random tokens as distractors. User was required to identify correct tokens and drag them to the positions on the screen where they remember seeing them (see FIG. 12.B). Feedback on spatial accuracy was given by displaying correct tokens as semitransparent overlays (see FIG. 12.C).

### Session Feedback

At the end of each session, Memoride produced a session feedback screen which included a graph of speed over time, numeric metrics representing the average speed, longest hold, number of recognized tokens and average token position error (in pixels), and a bar graph of four speed zones (0-5, 5-10, 10-15, 15-20 km/h).

### Experimental Protocol

The experimental protocol involved a total of 26 sessions: one pre-training session, 24 sessions of SDAN training, and one post-training session. Pre- and post-training sessions were administered in-person during which participants performed cognitive tests and recorded 2 minutes of eyes open and 2 minutes of eyes-closed resting state EEG with same BrainLink headsets. The first SDAN training session was performed in person at the laboratory as a way of onboarding participants to the technology. Participants were not provided with instructions and suggestions for SDAN training because of the algorithmic randomization in the control group. Any definitive strategy would necessarily mislead control subjects in at least some of the sessions. Participants were sent home equipped with an iPad and a headset, with instructions to perform the remaining training sessions twice daily over the period of up to 3 weeks, until full 24 sessions were completed. Research staff were provided with Memoride's web dashboard from which they could remotely provide tech support in-between training sessions. No support or supervision was given during active training.

### Cognitive Tests for Measuring Transfer Effects

During pre- and post-training sessions, participants performed a battery neurocognitive tests including an Attention Network Task (ANT), a verbal fluency test (FAS) and an N-Back memory task (NBACK).

ANT involves manipulation of cue and flanker type and allows the calculation of response time (RT) difference scores that are assumed to represent different aspects of attentional control. The tests consisted of a series of trials where a row of five horizontal black lines, with arrowheads pointing either left or right was presented. The target was a left or right facing arrowhead at the center. The target was flanked on either side by a) Congruent condition (two arrows in the same direction as the target); b) by Incongruent condition (two arrows in the opposite direction of the target), or c) by neutral condition (two lines on each side of the target). Participants had to identify the direction of the target (the central arrow) by pressing left or right arrow keys. The target position was randomized between the top and bottom of the screen. Each target was preceded by a cue screen which showed a crosshair in the center and: a spatial cue (single star at the top or bottom, indicating the location of the upcoming target), double cue (two stars, one at the bottom, and one at the top) and no cue. Besides standard measures of accuracy and response times (RT) for each cue-target combination, three attentional networks were also measured: Orienting Score (the difference in RT between Spatial Cue trials and No Cue and Double Cue trials), Alerting Score (the difference in RT between No Cue and all other cue types, regardless of target) and Conflict Score (the difference in RT between Incongruent and Congruent trials, regardless of cue type). To evaluate the effects of neuromodulation training on ANT task performance, an ANOVA was conducted with Group as between-subjects factor and Time (Pre-training, Post-training), Cue type, and Flanker type as within-subject factors. Moreover, to explore changes in three attentional networks, three further separate ANOVAs were performed: (1) for the alerting network, a 2 (Group)×2 (Alerting Score), (2) for the orienting network, a 2 (Group)×2 (Orienting Score), and (3) for the conflict network, a 2 (Group)×2 (Conflict Score). In post-hoc analyses a paired t-test was applied to examine Time effects (Pre vs Post) within each experimental group.

The FAS verbal fluency test (FAS) provides a measure of phonemic word fluency by requesting an individual to orally produce as many words as possible that begin with the letters F, A, and S within a 1 min time interval, for each letter. Verbal fluency is a cognitive function that facilitates information retrieval from memory. Successful retrieval requires executive control over cognitive processes such as selective attention, mental set shifting, internal response generation, and self-monitoring. The FAS is also considered to be a test of executive functions, including cognitive organization, initiation, maintenance of effort, and the ability to conduct a non-routine search for words based on a specific first letter, rather than the lexical definition. Words were recorded with timings using four 15 second blocks. Two scores were calculated: the total score FAS-Total which accounted for the total number of words produced, and the stratified score FAS-Stratified which was calculated by adding the number of words according to the following formula; F(1-15 sec)+A(15-30 sec)+S(30-45 sec).

In the NBACK task participants were presented with a series of visual stimuli and participants indicated whether it matched a stimulus that appeared n trials before. NBACK task is a classical test of working memory and attentional control. Varying n allows easy manipulation of working memory load and task difficulty. Two levels of n were used, n=1 (1-back) and n-2 (2-back). Two types of visual stimuli were presented, letters and colors, in 50 trials each. For example, in a 1-back task with letters as stimuli, participants tapped a button on the screen to indicate that the current letter was the same as the letter in the previous trial (n=1). In a 2-back task, participants tapped if the current letter was the same as the letter from the trial that was 2 trials ago (n=2). Accuracy and Response Time were measured for each n and stimulus type.

### EEG Analysis of Training Effects

In order to measure effects of training on EEG, average relative spectral power was measured in each session and the linear change in theta power across sessions was estimated. To normalize variability between subjects, average theta estimates were first z-scored within subject, across sessions. The slope of the line fitted to z-scored data was taken as an indicator of learning: the more positive the slope, the better the learning. A t-test was used to analyze learning slopes across the two groups.

### Results

### Transfer Effects

ANOVA was used to measure the difference between Pre and Post metrics across TNF (N=39) and RNF (N=27) groups for performance on cognitive tasks. Time (Pre, Post) was also analyzed within each group.

### Attention Network Task (ANT)

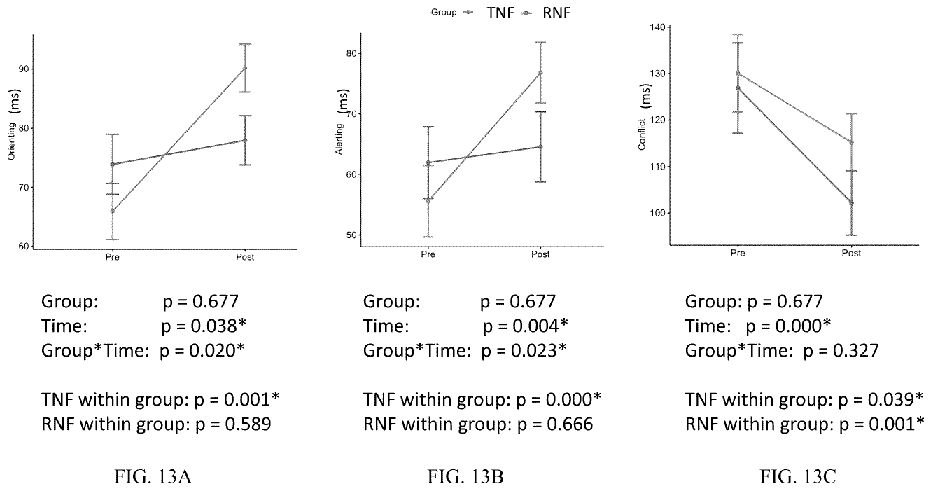

As shown in FIG. 13A-C, both groups showed time effects such that accuracy increased, and RT decreased from Pre to Post. Among the three attention networks, a significant effect of SDAN training was found, such that the TNF group showed a stronger Orienting effect compared to the RNF group. Only the TNF group became significantly more efficient in utilizing spatial cueing to produce faster responses (FIG. 13A). Similarly, the TNF group showed a stronger Alerting effect compared to the RNF group (FIG. 13B). Only the TNF group became significantly more efficient in utilizing cueing to produce faster responses. Conflict scores did not differ significantly between groups (FIG. 13C).

### FAS Verbal Fluency Test

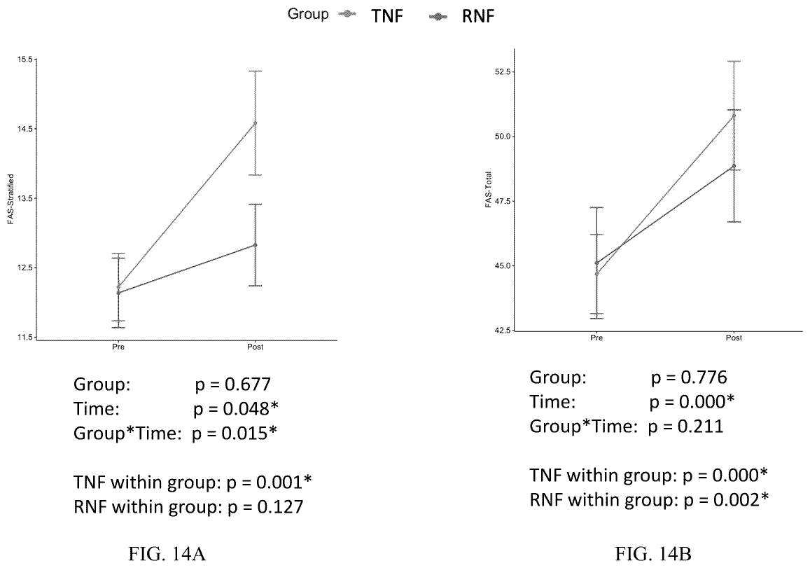

As shown in FIG. 14A-B, both groups showed time effects such that FAS-Stratified (FIG. 14A) and FAS-Total (FIG. 14B) increased from Pre to Post. There was a significant group-by-time effect of SDAN training, indicating that the TNF group showed a stronger increase in FAS-Stratified from Pre to Post. Only the TNF group showed a significant Pre-Post increase in FAS-Stratified. A similar trend was found in FAS-Total, although group-by-time interaction did not reach significance.

### NBACK Test

Neither the TNF group nor the RNF (control) group showed any Pre/Post effects in Accuracy for the 1-Back task. 2-Back Accuracy for combined stimuli increased significantly for the TNF group (p<0.05) from Pre to Post, but not for the RNF group. Group*time was not significant for 2-Back Accuracy. There was a significant group by time interaction for RT for pictures.

### EEG Training Effects

Learning slopes were observed to distinguish between the two groups. The average slope was 0.0281 in the TNF group and −0.0017 in the RNF group. Group difference was significant (p<0.04).

A correlation was also observed between learning slopes and performance on cognitive tasks, indicating that brain changes were driving the behavioral improvements.

### Conclusions

SDAN training produced expected EEG changes in the participants. Results on cognitive tests improved for both groups. Several measures, however, improved significantly more for the intervention group (TNF) compared to the active control group (RNF). In particular, FAS-Stratified, Alerting and Orienting network scores, 2-back accuracy, all showed robust group differences between the TNF and RNF groups. The inventor believes that this is the first study to have shown significant transfer effects associated with neurofeedback training using consumer-level EEG headsets and self-administered training.

### Discussion

A comparison of cognitive performance before and after neuromodulation training showed improved results for both groups. Since the Memoride application combined neuromodulation with light-weight cognitive training, some level of improvement may be attributed to the memory task, which was the same for both groups. For the TNF group, cognitive gains may also be attributed to consistent neuromodulation training.

Remarkably, effects were seen despite the neuromodulation training being largely performed outside of a clinical setting at home and unsupervised. Furthermore, the average age of study participants was 70 and they had to overcome a range of technological barriers, from learning to use an iPad, connecting headsets via Bluetooth, properly positioning the headset and all the way to troubleshooting and communicating remotely with technical support.

Nonetheless, in spite of these impediments, transfer effects were observed that were unique to the TNF group. Improvements that were significantly larger for the TNF group compared to the RNF group were also observed in a range of cognitive tests. TNF group showed stronger improvement in the areas of cognitive verbal fluency, alerting and orienting aspects of attention, executive control, and working memory.

### Example 2: Neurofeedback Training for Wellness with a Single Neuromodulation Marker Based on Weighted Parameters for Alpha and Theta Brainwaves

A study was conducted to in order to assess the use of neuromodulation therapy on wellness metrics. Neurofeedback was generated using a neuromodulation recipe that comprised a single neuromodulation marker and was based on statistical estimates (percentile values) of alpha and theta brainwave amplitudes with different weighting parameters for each brainwave.

### Experimental Design and Protocols

### Test Subjects

19 subjects were recruited into the study and asked to complete ˜20 sessions of neuromodulation training on a daily basis. Each subject was onboarded in-person to the neuromodulation platform and support was provided to the subjects remotely via an in-app chat for the duration of the study.

### Neuromodulation Platform

Each neuromodulation session started with a wellness survey that was designed to capture the subject's overall sense of wellbeing for the day and included the wellness metrics shown in Table 1. Subjects were asked to report on wellness metrics relating to the past 24 hours.

TABLE 1
Wellness metrics for self-evaluation prior to each therapy session

Wellness Metrics
Sleep (hours)
Stress (due to external circumstances)
Anxiety (internal response)
Imagination (rate 1-3)
Creativity (rate 1-3)
Last meal (how long ago-hours)
Food (how healthy, rating 1-3)
Alcohol (yes/no)
Stimulants (yes/no)
Mood/Emotions (overall positivity rating)
Energy (rate 1-5)
Productivity (rate 1-5)

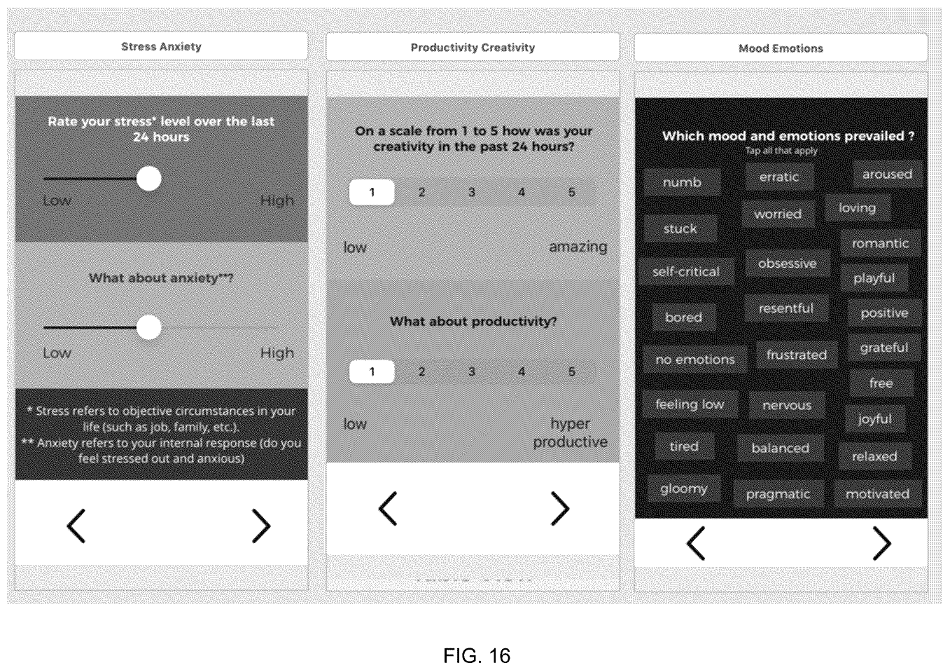

In order to measure Mood/Emotions, each subject picked words that he/she experienced the most from a list of possible choices e.g. “playful”, “balanced” or “pragmatic”. For processing, words were rated by “positivity” using numbers between −2 and 2, where −2 was given to more extreme negative words, and +2 for more extreme positive words like “joyful”. Each subject picked several words, and all the corresponding ratings were added together for an overall estimate of Mood/Emotions. Screenshots of the user interface used to determine the wellness metrics are shown in FIG. 16.

Neurofeedback was provided to each subject using the following neuromodulation recipe:

$$R = p ( r ) , where r = - 0.35 * p ( {sp}_{α} ) + 0.65 * p ( {sp}_{θ} )$$

Baseline distributions of the spectral power of alpha brainwaves (sp_{α}), and theta brainwaves (sp_{θ}) as well as for r (according to the above-noted neuromodulation recipe) were each determined at the start of each neuromodulation session for each subject. Baseline distributions were obtained during an introductory audio lesson that was played between 1.5 and 2 minutes preceding the onset of active neuromodulation training. These distributions were then used to determine percentile values p(sp_{α}), p(sp_{θ}) and p(r) which were then used to determine the neuromodulation maker R that was temporally smoothed and communicated to the user during the session by adjusting the music volume using the formulas and rules set out in Example 1.

The neuromodulation performance metrics identified in Table 2 were recorded for each subject across all sessions. Performance was re-evaluated every 60 seconds and automatic re-baselining was enforced if mean value of Rₛₘₒₒₜₕ was <45.

TABLE 2
Neuromodulation performance metrics

Neuromodulation Performance Metric
Zonetime (Percent time spent ”in the flow zone”)
Average (Average performance, avg_tp_score)
Gongs (Number of rewards for sustained flow, score)

### Data Analysis & Results

The 19 subjects each completed between 10 and 20 neuromodulation therapy sessions. For each subject and metric, the slope across training sessions was calculated and taken as a measure of the impact of the therapy sessions on the metric. The slope estimates linear change where a positive slope is indicative of an increase and a negative slope is indicative of a decrease.

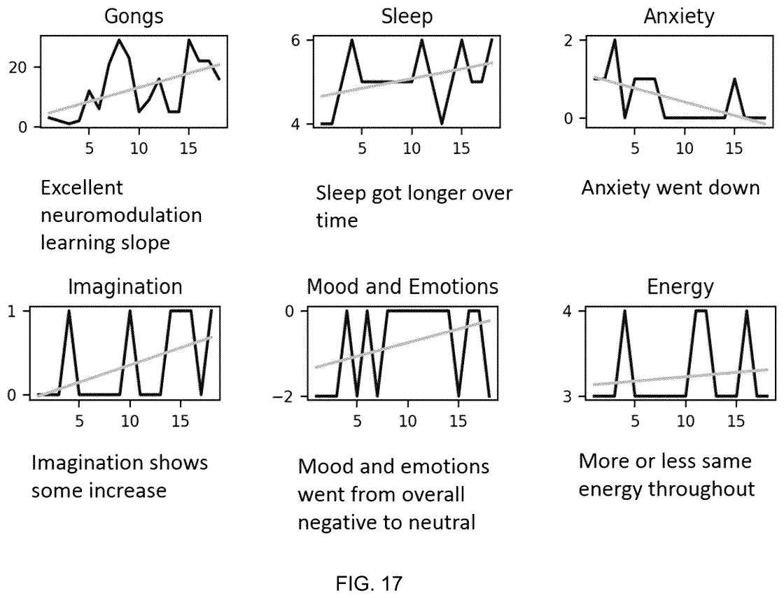

Exemplary raw data from a subject who completed 18 training sessions is shown in FIG. 17. The fitted slope shows the linear rate of change over the 18 sessions. Notably, the subject spent more time “in the zone” as the study went on and exhibited an excellent neuromodulation learning slope. The subject also reported an improvement in mood (positive slope) and decrease in anxiety (negative slope).

### Z-Scoring and Regressing Stress

Initial analysis of the study data showed that some subjects exhibited larger variability than others and the resulting metrics were therefore at different scales. Z-scoring was therefore used to remove this extraneous source of variability. Z-scoring was applied to the time series of each metric for each subject. Subsequently, a slope was fitted to the data based on the normalized time series. These slopes were averaged to produce group mean slope values, shown in Table 3.

In the neuromodulation application interface, stress was defined as a measure of external circumstances. Since stress generally has a strong impact on the wellbeing and it may have played a role during the study influencing the outcome of the other metrics. Accordingly, the effects of stress were regressed from all other metrics prior to fitting the slope in order to estimate the effects of neuromodulation therapy on the internal perception of wellbeing, without the influence of stress. The resulting group data is shown in Table 3.

TABLE 3
Raw group slopes for each metric, Z-scored and corrected for stress.

| | Raw | | Z-scored | | Z-scored w/o stress | |
|---|---|---|---|---|---|---|
| | Slope | P | Slope | P | Slope | P |
| Sleep | 0.016 | 0.33 | −0.017 | 0.4 | −0.016 | 0.33 |
| Stress | 0.004 | 0.95 | −0.004 | 0.98 | N/A | N/A |
| Anxiety | 0.018 | 0.21 | 0.032 | 0.23 | 0.015 | 0.45 |
| Imagination | 0.03 | 0.02* | 0.047 | 0.02* | 0.04 | 0.02* |
| Creativity | 0.017 | 0.17 | 0.035 | 0.08 | 0.032 | 0.04* |
| Last Meal | 0.063 | 0.49 | 0.011 | 0.42 | 0 | 0.97 |
| Food | 0.002 | 0.79 | −0.003 | 0.87 | 0.001 | 0.98 |
| Alcohol | 0.006 | 0.37 | −0.006 | 0.37 | −0.005 | 0.59 |
| Stimulants | −0.004 | 0.4 | −0.005 | 0.33 | −0.005 | 0.58 |
| Mood/ | 0.024 | 0.44 | 0.015 | 0.4 | 0.02 | 0.17 |
| Emotions | | | | | | |
| Energy | 0.006 | 0.72 | 0.007 | 0.6 | 0.011 | 0.63 |
| Productivity | 0.034 | 0.15 | 0.043 | 0.12 | 0.039 | 0.04* |
| Zone Time | 0.755 | 0 | 0.066 | 0 | 0.048 | 0.01* |
| Average | 0.005 | 0 | 0.065 | 0 | 0.048 | 0.01* |
| Gongs | 0.877 | 0 | 0.092 | 0 | 0.072 | 0 |

### Correlations Between Metrics

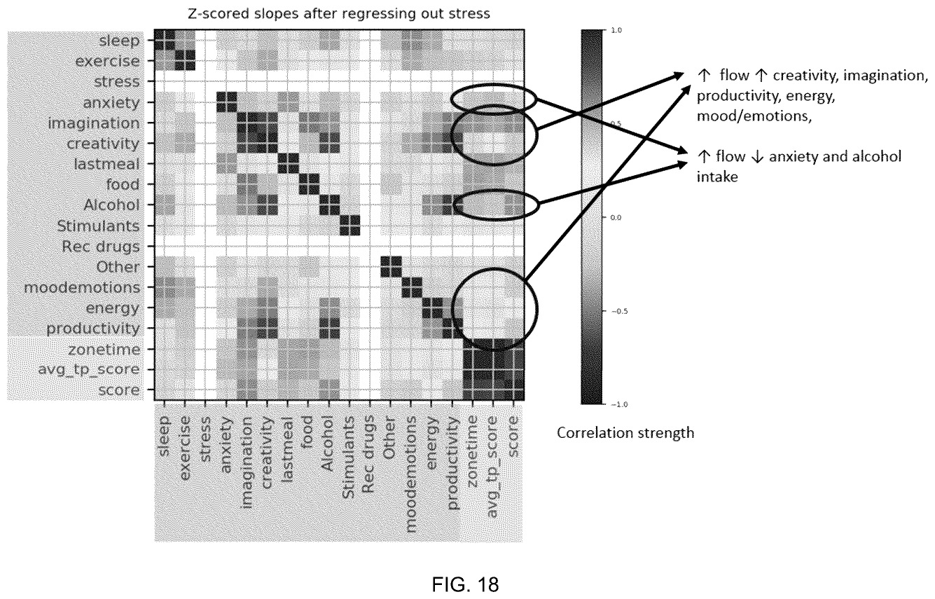

FIG. 18 shows the correlations between the Z-scored slopes (after regressing out stress).

### Conclusions

Over the course of the study a significant increase in Imagination, Creativity and Productivity metrics were observed at the group (n=19) level. Furthermore, a significant increase in all three neuromodulation performance metrics was observed, demonstrating that the subjects improved their neuromodulation skills over time. As shown in FIG. 18, better flow performance is related to an increase in creativity, imagination, productivity, energy, mood/emotions, and at the same time better flow performance is related to a decrease in anxiety and alcohol intake. Overall, this demonstrates that better flow performance using the neuromodulation platform is associated with better overall wellness.

This study also demonstrates that the use of a neuromodulation recipe that combines multiple weighted EEG measures into a single neuromodulation marker is effective for neuromodulation therapy and that users are able to improve their neuromodulation performance over time.

All referenced publications, patents and patent applications are herein incorporated by reference in their entirety to the same extent as if each individual publication, patent or patent application was specifically and individually indicated to be incorporated by reference in its entirety.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

### REFERENCES

Ali JI, Viczko J, Smart CM. Efficacy of Neurofeedback Interventions for Cognitive Rehabilitation Following Brain Injury: Systematic Review and Recommendations for Future Research. J Int Neuropsychol Soc. 2020;26(1):31-46. doi:10.1017/S1355617719001061
Allred SR, Radonjic A, Gilchrist AL, Brainard DH. Lightness perception in high dynamic range images: local and remote luminance effects. J Vis. 2012;12(2):10.1167/12.2.7 7. Published 2012 Feb 8. doi:10.1167/12.2.7
Ando, M., Kukihara, H., & Ide, N. (2017). Executive function measured by Stroop test and mood for elderly people in a facility for the elderly. Clinical case reports and reviews, 3.
Baier B, Kleinschmidt A, Müller NG. Cross-modal processing in early visual and auditory cortices depends on expected statistical relationship of multisensory information. J Neurosci. 2006;26(47):12260-12265. doi:10.1523/JNEUROSCI. 1457-06.2006
Balconi, Michela et al. Conscious, Pre-Conscious And Unconscious Mechanisms In Emotional Behaviour. Some Applications To The Mindfulness Approach With Wearable Devices. Applied Sciences 7.12 (2017): 1280. Web. 15 June 2020
Balconi M, Crivelli D, Angioletti L. Efficacy of a Neurofeedback Training on Attention and Driving Performance: Physiological and Behavioral Measures. Front Neurosci. 2019;13:996. Published 2019 Sep 18. doi:10.3389/fnins.2019.00996
Brandmeyer T, Delorme A. Closed-Loop Frontal Midlineθ Neurofeedback: A Novel Approach for Training Focused-Attention Meditation. Front Hum Neurosci. 2020;14:246. Published 2020 Jun 30. doi:10.3389/fnhum.2020.00246
Coben R, Hammond DC, Arns M. 19 Channel Z-Score and LORETA Neurofeedback: Does the Evidence Support the Hype?. Appl Psychophysiol Biofeedback. 2019;44(1):1-8. doi:10.1007/s10484-018-9420-6
Corcoran AW, Alday PM, Schlesewsky M, Bornkessel-Schlesewsky I. Toward a reliable, automated method of individual alpha frequency (IAF) quantification. Psychophysiology. 2018;55(7):e13064. doi:10.1111/psyp. 13064
Cortese S, Ferrin M, Brandeis D, et al. Neurofeedback for Attention-Deficit/Hyperactivity Disorder: Meta-Analysis of Clinical and Neuropsychological Outcomes From Randomized Controlled Trials. J Am Acad Child Adolesc Psychiatry. 2016;55(6):444-455. doi:10.1016/j.jaac.2016.03.007
Eckner JT, Lipps DB, Kim H, Richardson JK, Ashton-Miller JA. Can a clinical test of reaction time predict a functional head-protective response?. Med Sci Sports Exerc. 2011;43(3):382-387. doi:10.1249/MSS.0b013e3181f1cc51
Enriquez-Geppert S, Huster RJ, Herrmann CS. EEG-Neurofeedback as a Tool to Modulate Cognition and Behavior: A Review Tutorial. Front Hum Neurosci. 2017;11:51. Published 2017 Feb 22. doi:10.3389/fnhum.2017.00051
Evans KK, Treisman A. Natural cross-modal mappings between visual and auditory features. J Vis. 2010;10(1):1-12. Published 2010 Jan 12. doi:10.1167/10.1.6
Foroughi CK, Monfort SS, Paczynski M, Mcknight PE, Greenwood PM. Placebo effects in cognitive training. Proc Natl Acad Sci U S A. 2016;113(27):7470-7474. doi:10.1073/pnas. 1601243113
Ghodrati M, Zavitz E, Rosa MGP, Price NSC. Contrast and luminance adaptation alter neuronal coding and perception of stimulus orientation. Nat Commun. 2019;10(1):941. Published 2019 Feb 26. doi:10.1038/s41467-019-08894-8
Guez J, Rogel A, Getter N, et al. Influence of electroencephalography neurofeedback training on episodic memory: a randomized, sham-controlled, double-blind study. Memory. 2015;23(5):683-694. doi:10.1080/09658211.2014.921713
Hagenaars SP, Cox SR, Hill WD, et al. Genetic contributions to Trail Making Test performance in UK Biobank. Mol Psychiatry. 2018;23(7):1575-1583. doi:10.1038/mp.2017.189
Hanslmayr S, Sauseng P, Doppelmayr M, Schabus M, Klimesch W. Increasing individual upper alpha power by neurofeedback improves cognitive performance in human subjects. Appl Psychophysiol Biofeedback. 2005;30(1):1-10. doi:10.1007/s10484-005-2169-8
Kadosh KC, Staunton G. A systematic review of the psychological factors that influence neurofeedback learning outcomes. Neuroimage. 2019;185:545-555. doi:10.1016/j.neuroimage.2018.10.021
Kerson C, deBeus R, Lightstone H, et al. EEG Theta/Beta Ratio Calculations Differ Between Various EEG Neurofeedback and Assessment Software Packages: Clinical Interpretation. Clin EEG Neurosci. 2020;51(2):114-120. doi:10.1177/1550059419888320
Kochan NA, Bunce D, Pont S, Crawford JD, Brodaty H, Sachdev PS. Is intraindividual reaction time variability an independent cognitive predictor of mortality in old age? Findings from the Sydney Memory and Ageing Study. PLOS One. 2017;12(8): e0181719. Published 2017 Aug 9. doi:10.1371/journal.pone.0181719

Mladenović J, Mattout J, Lotte F. A Generic Framework for Adaptive EEG-Based BCI Training and Operation. In: Nam CS, Nijholt A, Lotte F, editors. Brain-Computer Interfaces Handbook: Technological and Theoretical Advances. Taylor & Francis; 2018.
Omejc N, Rojc B, Battaglini PP, Marusic U. Review of the therapeutic neurofeedback method using electroencephalography: EEG Neurofeedback. Bosn J Basic Med Sci. 2019;19(3):213-220. Published 2019 Aug 20. doi:10.17305/bjbms.2018.3785
Patching GR, Quinlan PT. Cross-modal integration of simple auditory and visual events. Percept Psychophys. 2004;66(1):131-140. doi:10.3758/bf03194867

Rammsayer TH, Borter N, Troche SJ. Visual-auditory differences in duration discrimination of intervals in the subsecond and second range. Front Psychol. 2015;6:1626. Published 2015 Oct 26. doi:10.3389/fpsyg.2015.01626
Reynolds JN, Wickens JR. Dopamine-dependent plasticity of corticostriatal synapses. Neural Netw. 2002;15(4-6):507-521. doi:10.1016/s0893-6080 (02)00045-x
Rogala J, Jurewicz K, Paluch K, Kublik E, Cetnarski R, Wróbel A. The Do's and Don'ts of Neurofeedback Training: A Review of the Controlled Studies Using Healthy Adults. Front Hum Neurosci. 2016;10:301. Published 2016 Jun 17. doi:10.3389/fnhum.2016.00301
Rogers JM, Johnstone SJ, Aminov A, Donnelly J, Wilson PH. Test-retest reliability of a single-channel, wireless EEG system. Int J Psychophysiol. 2016;106:87-96. doi:10.1016/j.ijpsycho.2016.06.006
Schabus M, Griessenberger H, Gnjezda MT, Heib DPJ, Wislowska M, Hoedlmoser K. Better than sham? A double-blind placebo-controlled neurofeedback study in primary insomnia. Brain. 2017;140(4):1041-1052. doi:10.1093/brain/awx011
Sitaram R, Ros T, Stoeckel L, et al. Closed-loop brain training: the science of neurofeedback [published correction appears in Nat Rev Neurosci. 2019 May;20(5):314]. Nat Rev Neurosci. 2017;18(2):86-100. doi:10.1038/nrn.2016.164
Strehl U. What learning theories can teach us in designing neurofeedback treatments. Front Hum Neurosci. 2014;8:894. Published 2014 Nov. 6. doi:10.3389/fnhum.2014.00894
Wang JR, Hsieh S. Neurofeedback training improves attention and working memory performance. Clin Neurophysiol. 2013;124(12):2406-2420. doi:10.1016/j.clinph.2013.05.020
Xiong S, Cheng C, Wu X, Guo X, Yao L, Zhang J. Working memory training using EEG neurofeedback in normal young adults. Biomed Mater Eng. 2014;24(6):3637-3644. doi:10.3233/BME-141191
Zurrón M, Lindín M, Galdo-Alvarez S, Diaz F. Age-related effects on event-related brain potentials in a congruence/incongruence judgment color-word Stroop task. Front Aging Neurosci. 2014;6:128. Published 2014 Jun 17. doi:10.3389/fnagi.2014.00128

I claim:

1. A neuromodulation device for providing a neuromodulation session for a subject, the neuromodulation device positioned proximate to the subject's head, the device comprising:
a sensor for receiving a first sensor signal measuring the subject's brain activity during a first time interval;
a memory, comprising:
a database comprising a subject baseline distribution of a first neuromodulation marker obtained from the subject, subject cognitive data comprising one or more cognitive test results, subject input data, and a pre-determined reference distribution of the first neuromodulation marker, the subject baseline distribution based on historical subject sensor data, the pre-determined reference distribution based on aggregated population data;
an output device for providing a neuromodulation user interface of the neuromodulation session to the subject;
a processor coupled to the sensor and the memory, and being configured to:
receive the first sensor signal from the sensor;
determine at the processor, the first neuromodulation marker based on one or more psychophysiological parameters of the first sensor signal, the subject baseline distribution, the subject cognitive data, and the pre-determined reference distribution, wherein the first neuromodulation marker provides a personalized neuromodulation metric for the subject; and
generate the neuromodulation user interface for the subject, the neuromodulation user interface providing a sensory feedback output determined based on the first neuromodulation marker.

2. The device of claim 1 wherein the processor is further configured to:
determine the first neuromodulation marker based on a machine learning model.

3. The device of claim 1 wherein the processor is further configured to:
determine an amplitude value of each of the one or more psychophysiological parameters;
determining a statistical estimate of the amplitude value of each of the one or more psychophysiological parameters relative to the pre-determined reference distribution of the amplitude value of each of the one or more psychophysiological parameters, the reference distribution of the amplitude value of each of the one or more psychophysiological parameters; and
determine the first neuromodulation marker based on the statistical estimate of the amplitude value of each of the one or more psychophysiological parameters.

4. The device of claim 3, wherein
the sensor receives from the neuromodulation device, a second sensor signal of the subject's brain activity during a second time interval;
the processor is further configured to:
determine the one or more psychophysiological parameters based on the second sensor signal;
determine an amplitude value of each of the one or more psychophysiological parameters for the second sensor signal;
determine a second neuromodulation marker based on one or more amplitude values of the one or more psychophysiological parameters for the second sensor signal, and
the output device provides the neuromodulation user interface for the subject by changing the sensory feedback output based on the second neuromodulation marker.

5. The device of claim 1, wherein the sensor comprises an electroencephalogram (EEG) sensor, a functional magnetic resonance imaging (fMRI) sensor, a magnetoencephalography (MEG), or a functional Near Infrared Spectroscopy (fNIRS) sensor.

6. The device of claim 5, wherein the processor is further configured to:
select the one or more psychophysiological parameters from the group consisting of spectral power, entropy, phase synchronization, asymmetry, and coherence between different electrode sites.

7. The device of claim 6, wherein the processor is further configured to select the one or more psychophysiological parameters based on user input.

8. The device of claim 7 wherein the processor is configured to determine spectral power values of the one or more psychophysiological parameters from one or more spectral frequency bands of the first sensor signal or the second sensor signal.

9. The device of claim 7, wherein the one or more spectral frequency bands comprise at least one of an alpha band, a beta band, a delta band, a gamma band, a theta band, and the first neuromodulation marker or the second neuromodulation marker are determined based on at least one frequency band selected from the group consisting of the alpha band, the beta band, the gamma band, and the theta band.

10. The device of claim 1, wherein the output device comprises at least one selected from the group consisting of a display device, an audio output device, and a tactile device and the output signal comprises at least one selected from the group consisting of a visual output displayed on the display device, an audio output to the audio output device, and a tactile output to the tactile device.

11. The device of claim 10, wherein the processor is configured to:
provide a video game application to the subject using the neuromodulation device, the video game comprising the sensory feedback output;
provide an interactive soundscape application to the subject using the neuromodulation device, the interactive soundscape application comprising the sensory feedback output;
provide a meditation application to the subject using the neuromodulation device, the meditation application comprising the sensory feedback output;
provide a cognitive testing application to the subject using the neuromodulation device and storing subject cognitive testing data in the database, the subject cognitive testing data comprising one or more cognitive test results; or
provide a self-evaluation application to the subject using the neuromodulation device and storing subject self-evaluation data in the database.

12. A neuromodulation device for providing a neuromodulation session for a subject, the neuromodulation device positioned proximate to the subject's head, the device comprising:
a sensor for receiving a first sensor signal measuring the subject's brain activity during a first time interval;
a memory, comprising:
a database comprising a subject baseline distribution of a first neuromodulation marker obtained from the subject, subject cognitive data comprising one or more cognitive test results, subject input data, and a pre-determined reference distribution of the first neuromodulation marker, the subject baseline distribution based on historical subject sensor data, the pre-determined reference distribution based on aggregated population data;
a processor coupled to the sensor and the memory, and being configured to:
receive the first sensor signal from the sensor;
determine at the processor, the first neuromodulation marker based on one or more psychophysiological parameters of the first sensor signal, the subject baseline distribution, the subject cognitive data, and the pre-determined reference distribution, wherein the first neuromodulation marker provides a personalized neuromodulation metric for the subject;
generate the neuromodulation user interface for the subject, the neuromodulation user interface providing a sensory feedback output determined based on the first neuromodulation marker;
transmit the neuromodulation user interface for the subject to an output device.

13. The device of claim 12, wherein
the sensor receives from the neuromodulation device, a second sensor signal of the subject's brain activity during a second time interval;
the processor is further configured to:
determine the one or more psychophysiological parameters based on the second sensor signal;
determine an amplitude value of each of the one or more psychophysiological parameters for the second sensor signal;
determine a second neuromodulation marker based on one or more amplitude values of the one or more psychophysiological parameters for the second sensor signal, and
the output device provides the neuromodulation user interface for the subject by changing the sensory feedback output based on the second neuromodulation marker.

14. The device of claim 12, wherein the sensor comprises an electroencephalogram (EEG) sensor, a functional magnetic resonance imaging (fMRI) sensor, a magnetoencephalography (MEG), or a functional Near Infrared Spectroscopy (fNIRS) sensor.

15. The device of claim 14 wherein the output device is in wireless communication with the neuromodulation device.

16. The device of claim 15 wherein the output device is connected to the neuromodulation device using at least one selected from the group of a Bluetooth® connection and an 802.11x connection.

17. The device of claim 16 wherein the output device is an Android® device.

18. The device of claim 16 wherein the output device is an iOS® device.

19. A neuromodulation system for providing a neuromodulation session to a subject, the neuromodulation system comprising:
a neuromodulation device positioned proximate to the subject's head during the neuromodulation session, and
an output device coupled to the neuromodulation device, the output device configured to provide a neuromodulation user interface for the subject,
a database, comprising:
a subject baseline distribution of a first neuromodulation marker obtained from the subject, subject cognitive data comprising one or more cognitive test results, subject input data and a pre-determined reference distribution of the first neuromodulation marker, the subject baseline distribution being based on historical sensor data of the subject, the pre-determined reference distribution based on an aggregated population data;
the neuromodulation device comprising:
a sensor for receiving a first sensor signal measuring the subject's brain activity during a first time interval during the neuromodulation session;
a processor coupled to the memory and the sensor, the processor being configured to:
receive the first sensor signal from the sensor;
determine at the processor, the first neuromodulation marker based on one or more psychophysiological parameters of the first sensor signal, the subject baseline distribution, the subject cognitive data, and the pre-determined reference distribution, wherein the first neuromodulation marker provides a personalized neuromodulation metric for the subject; and
generate a neuromodulation user interface signal for the subject, the neuromodulation user interface signal providing a sensory feedback output determined based on the first neuromodulation marker; and
transmit the neuromodulation user interface signal to an output device coupled to the neuromodulation device, and
wherein the output device is configured to receive the neuromodulation user interface signal and generate the neuromodulation user interface for display to the subject.

20. The system of claim 19 wherein the database comprises a remote database in network communication with the neuromodulation device.
